(19) United States
(12) Patent Application Publication
(10) Pub. No.: US 2015/0224203 A1
(43) Pub. Date: Aug. 13, 2015

(54) Factor VIII Conjugates

(71) Applicant: NOVO NORDISK A/S, Bagsvaerd (DK)

(72) Inventors: Carsten Behrens, Koebenhavn (DK); Paul L. DeAngelis, Edmond, OK (US); Friedrich Michael Haller, Norman, OK (US)

(21) Appl. No.: 14/620,587

(22) Filed: Feb. 12, 2015

(30) Foreign Application Priority Data
Feb. 12, 2014 (EP) 14154876.8

(51) Int. Cl. A61K 47/48; G01N 33/86; A61K 38/37

(52) U.S. Cl. CPC A61K 47/4823; A61K 38/37; G01N 33/86

(57) ABSTRACT

The present invention relates to FVIII conjugated to heparosan (HEP) polymers, methods for the manufacture thereof and uses of such conjugates. The resultant conjugates may be used in the treatment or prevention of bleeding disorders such as haemophilia.

### FIELD OF THE INVENTION

The present invention relates to conjugates between blood coagulation Factor VIII and heparosan polymers and uses thereof.

### CROSS-REFERENCE TO RELATED APPLICATIONS

This application claims priority under 35 U.S.C. §119 of European Patent Application 14154876.8, filed Feb. 12, 2014; the contents of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Feb. 11, 2015, is named 130085WO01_SeqList.txt and is 21,520 bytes in size.

### BACKGROUND

Protein replacement therapy by IV administration of FVIII is currently used for treating patients suffering from haemophilia A. Current treatment recommendations are moving from traditional on-demand treatment towards prophylaxis. The circulatory half-life of endogenous FVIII is 12-14 hours and prophylactic treatment is thus to be performed several times a week in order to obtain a virtually symptom-free life for the patients. For many patient, especially children, IV administration is associated with significant inconvenience and/or pain as well as risk of infections, in particular in connection with catheters. There is thus a need in the art for FVIII compounds having a significantly prolonged circulatory half-life in order to reduce the frequency of FVIII IV administrations.

Conjugation of FVIII with side chains of polymeric nature (e.g. PEG) in order to prolong circulatory half-life is known in the art. Conjugation of half-life extending moieties—e.g. in the form of a hydrophilic polymer—with a peptide or polypeptide can be carried out by enzymatic methods. These methods can be selective, requiring the presence of specific peptide consensus motives in the protein sequence, or the presence of post translational moieties such as glycans. Selective enzymatic methods for modifying N- and O-glycans on blood coagulation factors have been described. For example, chemically modified sialic acid substrates (Malmstrøm, J, Anal Bioanal Chem. 2012; 403:1167-1177) have been described that can be used to glycoPEGylate Factor Vila on N-glycans using sialyltransferase ST3GaIIII (Stennicke, H R. et al. Thromb Haemost. 2008 November; 100(5):920-8), and on O-glycans on Factor VIII using ST3GaII (Stennicke, H R. et al., Blood. 2013 Mar. 14; 121(11):2108-16).

A common feature of the selective enzymatic methods is the use of a modified sialic acid substrate, glycyl sialic acid cytidine monophosphate (GSC), as well as the chemical acylation of GSC with the half-life extending moieties.

For example, PEG polymers activated as nitrophenyl- or N-hydroxy-succinimide esters can be acylated onto the glycyl amino group of GSC to create a PEG substituted sialic acid substrate that can be enzymatically transferred to the N- and 0-glycans of glycoproteins (cf. WO2006127896, WO2007022512, US2006040856). In a similar way, fatty acids can be acylated onto the glycyl amino group of GSC using N-hydroxy-succinimide activated ester chemistry (WO2011101277).

Common methods for linking half-life extending moieties such as carbohydrate polymers (e.g., heparosan) to glycoproteins such as FVIII comprise oxime, hydrazone or hydrazide bond formation. WO2006094810 describes methods for attaching hydroxyethyl starch polymers to glycoproteins such as erythropoietin that circumvent the problems connected to using activated ester chemistry. In these methods, hydroxyethyl starch and erythropoietin are individually oxidized with periodate on the carbohydrate moieties, and the reactive carbonyl groups ligated together using bis-hydroxylamine linking agents. The method will create hydroxyethyl starch linked to the erythropoietin via oxime bonds.

Similar oxime based linking methodology can be imagined for attaching carbohydrate polymers to GSC (see for example WO11101267), however, as such oxime bonds are known to exist in both syn- and anti-isomer forms, the linkage between the polymer and the protein will contain both syn- and anti-isomer combinations. Such isomer mixtures are usually not desirable in proteinaceous medicaments, such as FVIII, that are used for long term repeat administration since the linker inhomogeneity may pose a risk for antibody generation.

The above mentioned methods have further disadvantages. In the oxidative process required for activating the glycoprotein, parts of the carbohydrate residues are chemically cleaved and the carbohydrates will therefore not be present in intact form in the final conjugate. The oxidative process furthermore will generate product heterogeneity as the oxidating agent, i.e., periodate, in most cases is unspecific with regard to which glycan residue is oxidized. Both product heterogeneity and the presence of non-intact glycan residues in the final drug conjugate may impose immunogenicity risks.

Alternatives for linking carbohydrate polymers to glycoproteins, such as FVIII, involve the use of maleimide chemistry (WO2006094810). For example, the carbohydrate polymer can be furnished with a maleimido group, which selectively can react with a sulfhydryl group on the target protein. The linkage will then contain a cyclic succinimide group.

However, the inventors have found that previously published methods are not suited for attaching highly functionalized half-life extending moieties such as carbohydrate polymers to GSC.

### SUMMARY OF THE INVENTION

Described herein are novel heparosan-Factor VIII (HEP-FVIII) conjugates, methods for producing the conjugates, pharmaceutical compositions comprising the conjugates as well as use of the conjugates. The described preparation and properties of novel FVIII-heparosan polymer (HEP) molecules/conjugates contemplate various linker moieties. These conjugates provide certain advantages in relation to, for example, relative simplicity of the conjugation process. Advantageously, the described conjugates and methods have improved physical and/or chemical stability of side chains and/or linkers. Other advantages relate to homogenous products. Other advantages relate to advantageous assayability in assays, such as e.g. activated partial thromboplastin time (aPTT) assays, wherein relatively reliable and reproducible results can be obtained with the conjugates of the present invention. Other advantages relate to viscosity of liquid/aqueous solutions comprising conjugates prepared according to the described methods.

Various embodiments described herein provide conjugated FVIII compounds as well as conjugation methods, wherein FVIII is linked such that a stable and isomer free conjugate is obtained. FVIII conjugates obtained by or obtainable by the methods described herein as well as uses thereof are also provided.

The conjugates described herein are protected by a biodegradable half-life extending moiety in the form of heparosan (HEP) which extends the in vivo half-life of Factor VIII (FVIII). In some embodiments the HEP-FVIII polypeptide conjugate described herein has increased circulation half-life compared to an unconjugated FVIII polypeptide; or increased functional half-life compared to an unconjugated FVIII polypeptide.

In some embodiments the described HEP-FVIII conjugate has increased mean residence time compared to an unconjugated FVIII polypeptide; or increased functional mean residence time compared to an unconjugated FVIII polypeptide.

Moreover, in some embodiments the conjugates show improved performance compared to similar PEGylated FVIII variants in aPTT assays.

In one embodiment, the polymer may have an average size between approximately 5 and approximately 150 kDa, such as between approximately 35 and 45 kDa.

Also, the HEP-FVIII conjugates described herein can be produced using a linker which has improved properties (e.g., stability). In one embodiment, HEP-FVIII conjugates are provided wherein the HEP moiety is linked to FVIII in such a way that a stable and isomer free conjugate is obtained. In one embodiment, the HEP polymer is linked to FVIII using a chemical linker comprising 4-methylbenzoyl moiety connected to a sialic acid derivative such as glycyl sialic acid cytidine monophosphate (GSC).

The HEP-FVIII conjugates described herein are useful in the treatment of coagulopathy and in particular prophylactic treatment of haemophilia A.

### BRIEF DESCRIPTION OF DRAWINGS

FIG. 1: Functionalization of glycyl sialic acid cytidine monophosphate (GSC) with a benzaldehyde group. GSC is acylated with 4-formylbenzoic acid and subsequently reacted with heparosan (HEP-)amine by a reductive amination reaction.

FIG. 2: Functionalization of heparosan (HEP) polymer with a benzaldehyde group and subsequent reaction with glycyl sialic acid cytidine monophosphate (GSC) in a reductive amination reaction.

FIG. 3: Functionalization of glycyl sialic acid cytidine monophosphate (GSC) with a thio group and subsequent reaction with a maleimide functionalized heparosan (HEP) polymer.

FIG. 4: Heparosan (HEP)—glycyl sialic acid cytidine monophosphate (GSC).

FIG. 5: FVIII-HEP linker as described herein linked to amino acid residue Ser750 of Factor VIII (SEQ ID NO 1).

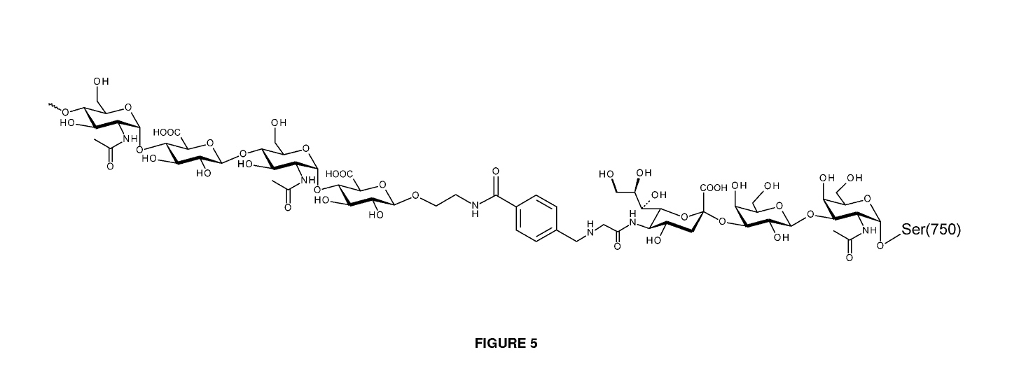

FIG. 6: Reaction of an asialo FVIII glycoprotein with HEP-GSC in the presence of sialyltransferase.

### BRIEF DESCRIPTION OF THE SEQUENCES

SEQ ID NO: 1: The amino acid sequence of wild-type human Factor VIII.

SEQ ID NO: 2: A 21 amino acid residue sequence (L) linking FVIII light chain (FVIII-LC) and FVIII heavy chain (FVIII-HC).

SEQ ID NO: 3: A 20 amino acid residue sequence (L) linking FVIII light chain (FVIII-LC) and FVIII heavy chain (FVIII-HC).

SEQ ID NO: 4: A 20 amino acid residue sequence (L) linking FVIII light chain (FVIII-LC) and FVIII heavy chain (FVIII-HC).

### DESCRIPTION

Described herein are novel heparosan -Factor FVIII polypeptide (HEP-FVIII) conjugates and preparation thereof. These conjugates provide biological properties superior to other conjugates known in the art.

Increasing the in vivo circulatory half-life of FVIII is desirable in order to reduce the frequency of FVIII administrations in haemophilia patients. The quality of the chemical linkage between the half-life extending moiety and FVIII is important for several reasons. From a manufacturing perspective, the type of linkage can affect isomer formation in the conjugate and is thus important in terms of product quality and regulatory considerations. From a storage perspective, the quality of the linkage affects the stability of the conjugate and is therefore important in terms of shelf-life. From a pharmacokinetic perspective, it is also important that the FVIII conjugate is stable in vivo in order to retain the desired functionality, such as long half-life.

There is thus a need in the art for methods of conjugating a half-life extending moiety to FVIII, wherein a stable and isomer free FVIII conjugate is obtained. A stable and isomer free linker for use in glycyl sialic acid cytidine monophosphate (GSC) based conjugation of FVIII is described herein.

The GSC starting material used herein can be synthesised chemically (Dufner, G. Eur. J. Org. Chem. 2000, 1467-1482) or it can be obtained by chemo-enzymatic routes as described in WO07056191.

The GSC structure is shown below:

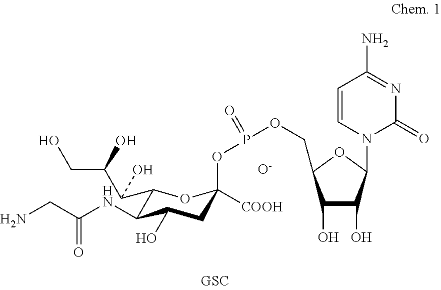

FVIII conjugates herein comprise a linker moiety comprising the following structure:

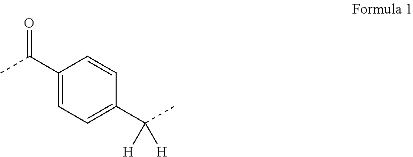

hereinafter also referred to as sublinker or sublinkage/sublinker—that connects a HEP-amine and GSC in one of the following ways:

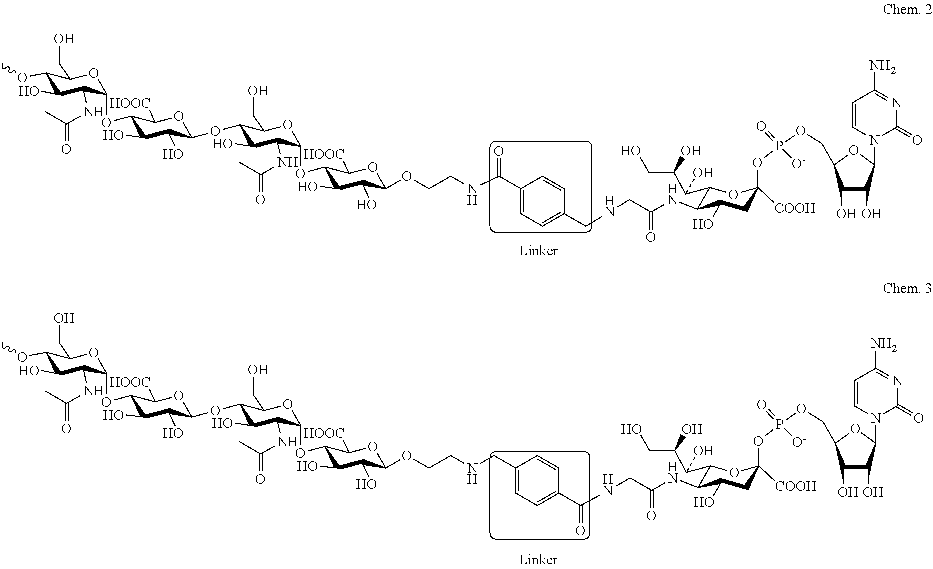

The highlighted 4-methylbenzoyl sublinker thus makes up part of the full linking structure linking the half-life extending moiety to FVIII. The highlighted 4-methylbenzoyl linker is a stable structure compared to alternatives, such as succinimide based linkers (prepared from maleimide reactions with sulfhydryl groups) since the latter type of cyclic linkage has a tendency to undergo hydrolytic ring opening when the conjugate is stored in aqueous solution for extended time periods (Bioconjugation Techniques, G. T. Hermanson, Academic Press, 3^{rd} edition 2013 p. 309). Even though the ring-opened succinimide linkage in this case (e.g. between HEP and sialic acid on FVIII) may remain intact, the ring opening reaction will add heterogeneity in form of regio- and stereo-isomers to the final FVIII conjugate composition.

One advantage associated with FVIII conjugates prepared according to the methods described herein is that a homogenous product is obtained where the tendency of isomer formation due to linker structure and stability is significantly reduced. Another advantage is that the FVIII conjugates can be produced in a simple process, preferably a one-step process. The 4-methylbenzoyl sublinkage, as used herein, between the half-life extending moiety and GSC is not able to form steno- or regio isomers. Isomer formation is undesirable due to the formation of a heterogeneous product and thereby an increased risk for unwanted immune responses in humans. Isomer formation is undesirable since presence of isomers can lead to a heterogeneous product and increase the risk for unwanted immune responses in humans.

### Heparosan

Heparosan (HEP) is a natural sugar (polysaccharide) polymer comprising (-GlcUA-1,4-GlcNAc-1,4-) repeats. It belongs to the glycosaminoglycan polysaccharide family and is a negatively charged polymer at physiological pH. HEP can be found in the capsule of certain bacteria but it is also found in higher vertebrate where it serves as precursor for the natural polymers heparin and heparan sulphate. Heparosan can be degraded by lysosomal enzymes such as N-acetyl-a-D-glucosaminidase (NAGLU) and β-glucuronidase (GUSB).

HEP polymers can be prepared by a synchronised enzymatic polymerisation reaction (US 20100036001) using heparan synthetase I from Pasturella multocida (PmHS1). This enzyme can be expressed in E. coli as a maltose binding protein (MBP) fusion constructs. Purified MBP-PmHS1 enzyme is able to produce monodisperse polymers in a synchronized, stoichiometrically controlled reaction, when it is added to an equimolar mixture of sugar nucleotides (e.g., GlcNAc-UDP and GlcUA-UDP). A trisaccharide initiator (e.g., GlcUA-GlcNAc-GlcUA) is used to prime the reaction, and polymer length is determined by the primer:sugar nucleotide ratios. The polymerization reaction typically runs until about 90% of the sugar nucleotides are consumed. Polymers are isolated from the reaction mixture by anion exchange chromatography, and subsequently freeze-dried into a stable powder. Processes for preparation of functional HEP polymers are described in US 201000036001. For example, US20100036001 lists aldehyde-, amine- and maleimide functionalized HEP reagents. A range of other functionally modified HEP derivatives are available using similar chemistry. HEP polymers used herein are initially produced with a primary amine handle at the reducing terminal according to methods described in US20100036001.

Amine-functionalized HEP polymers may be prepared according to US20100036001 and converted into heparosan benzaldehyde polymers by reaction with 4-formylbenzoic acid NHS ester. Heparosan benzaldehyde polymers may in a following step be coupled to the glycylamino group of GSC by a reductive amination reaction. The resulting HEP-GSC product can subsequently be enzymatically conjugated to FVIII using e.g., a sialyltransferase. The amine handle (reactive amine group) on HEP can be converted into a benzaldehyde handle (reactive aldehyde group) using N-hydroxysuccinimidyl 4-formylbenzoate according to the below scheme:

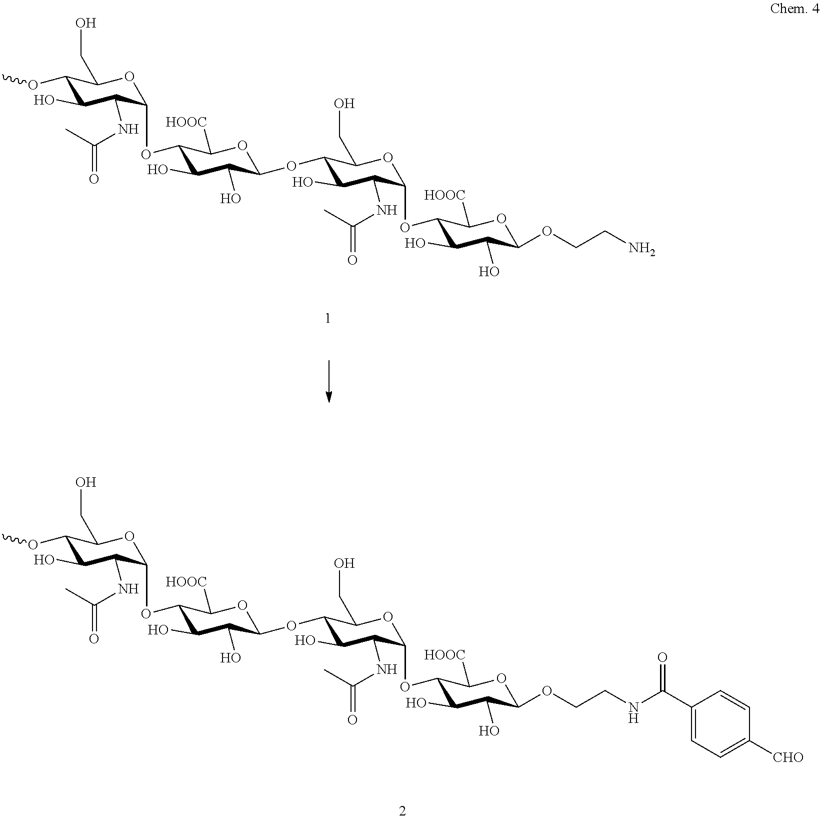

The conversion of HEP amine (1) to the 4-formylbenzamide compound (2) in scheme above may be carried out by reaction with acyl activated forms of 4-formylbenzoic acid. N-hydroxysuccinimidyl may be chosen as the acyl activating group, but a number of other acyl activation groups are known to the skilled person. Non-limited examples include 1-hydroxy-7-azabenzotriazole-, 1-hydroxy-benzotriazole-, pentafluorophenyl-esters as known from peptide chemistry. Benzaldehyde-modified HEP reagents can be kept stable for extended time periods when stored frozen (−80° C.) in dry form.

A heparosan polymer for use in the present invention is typically a polymer of the formula (-GlcUA-beta1,4-GlcNAc-alpha1,4-)ₙ. The size of the heparosan polymer may be defined by the number of repeats “n” in this formula. The number of said “n” repeats may be, for example, from 2 to about 5,000. The number of “n” repeats may be, for example 50 to 2,000 units, 100 to 1,000 units or 200 to 700 units. The number of “n” repeats may be 200 to 250 units, 500 to 550 units or 350 to 400 units. Preferably, “n” ranges from about 100 to about 125, such as e.g. 90-120, 95-115, or 94-116. Any of the lower limits of these ranges may be combined with any higher upper limit of these ranges to form a suitable range of numbers of units in the heparosan polymer.

The size of the heparosan polymer may be defined by its molecular weight. The molecular weight may be the average molecular weight for a population of heparosan polymer molecules, such as the weight average molecular mass. The heparosan polymer may have a molecular weight of, for example, 500 Da to 1,000 kDa. The molecular weight of the polymer may be 500 Da to 650 kDa, 5 kDa to 750 kDa, 10 kDa to 500 kDa, 15 kDa to 550 kDa, 25 kDa to 250 kDa or 50 kDa to 175 kDa.

The molecular weight may be selected at particular levels within these ranges in order to achieve a suitable balance between activity of the Factor VIII polypeptide and half-life of the conjugate. For example, the molecular weight of the polymer may be in a range selected from 5-15 kDa, 15-25 kDa, 25-35 kDa, 35-45 kDa, 45-55 kDa, 55-65 kDa, 65-75 kDa, 75-85 kDa, 85-95 kDa, 95-105 kDa, 105-115 kDa, 115-125 kDa, 125-135 kDa, 135-145 kDa, 145-155 kDa, 155-165 kDa or 165-175 kDa. More specific ranges of molecular weight may be selected. For example, the molecular weight may be 500 Da to 20 kDa, such as 1 kDa to 15 kDa, such as 5 kDa to 15 kDa, such as 8 kDa to 17 kDa, such as 10 kDa to 14 kDa such as about 12 kDa. The molecular weight may be 20 kDa to 35 kDa, such as 22 kDa to 32 kDa such as 25 kDa to 30 kDa, such as about 27 kDa. The molecular weight may be 35 to 65 kDa, such as 40 kDa to 60 kDa, such as 47 kDa to 57 kDa, such as 50 kDa to 55 kDa such as about 52 kDa. The molecular weight may be 50 to 75 kDa such as 60 to 70 kDa, such as 63 to 67 kDa such as about 65 kDa. The molecular weight may be 75 to 125 kDa, such as 90 to 120 kDa, such as 95 to 115 kDa, such as 100 to 112 kDa, such as 106 to 110 kDa such as about 108 kDa. The molecular weight may be 125 to 175 kDa, such as 140 to 165 kDa, such as 150 to 165 kDa, such as 155 to 160 kDa such as about 157 kDa, such as 20-157 kDa. The molecular weight may be 5 to 100 kDa, such as 10 to 60 kDa and such as 20 to 50 kDa.

Any of the lower limits of these ranges of molecular weight may be combined with any higher upper limit from these ranges to form a suitable range for the molecular weight of the heparosan polymer in accordance with the invention.

Molecular weight values as described herein in relation to size of the HEP polymer may in practise not be the exact size listed. Due to variations between individual batches during HEP polymer production, some variation in the HEP polymer size is to be expected. To encompass batch to batch variation, it is therefore to be understood, that a variation around +/−10%, 9%, 8%, 7%, 6%, 5%, 4%, 3%, 2% or 1% around target HEP polymer size is to be expected. For example, HEP polymer size of 40 kDa denotes 40 kDa+/−10%, e.g. 40 kDa could for example in reality mean that individual polymer sizes range from about 36-44 kDa, both falling within the ±10% range of 36 to 44 kDa of 40 kDa.

In connection with FVIII polypeptide conjugates herein, HEP offers a very flexible way of prolonging in vivo circulatory half-life since a wide ranges of HEP polymer sizes will result in a significantly improved half-life.

The heparosan polymer may have a narrow size distribution (e.g., monodisperse) or a broad size distribution (e.g., polydisperse). The level of polydispersity may be represented numerically based on the formula Mw/Mn, where Mw=weight average molecular mass and Mn=number average molecular weight. The polydispersity value using this equation for an ideal monodisperse polymer is 1. Preferably, a heparosan polymer for use in the present invention is monodisperse. The polymer may therefore have a polydispersity that is about 1, the polydispersity may be less than 1.25, preferably less than 1.20, preferably less than 1.15, preferably less than 1.10, preferably less than 1.09, preferably less than 1.08, preferably less than 1.07, preferably less than 1.06, and more preferably less than 1.05. The molecular weight size distribution of the heparosan may be measured by comparison with monodisperse size standards (HA Lo-Ladder™, Hyalose LLC) which may be run on agarose gels.

Alternatively, the size distribution of heparosan polymers may be determined by high performance size exclusion chromatography-multiangle laser light scattering (SEC-MALLS). Such a method can be used to assess the molecular weight and polydispersity of a heparosan polymer. Polymer size may be regulated in enzymatic methods of production. By controlling the molar ratio of heparosan acceptor chains to UDP sugar, it is possible to select a final heparosan polymer size that is desired.

### Methods for Preparing FVIII-HEP Conjugates

It is shown by the present inventors that it is possible to link a carbohydrate polymer, e.g. HEP, via a maleimido group to a thio-modified GSC molecule and transfer the reagent to an intact glycosyl groups on a glycoprotein such as FVIII by means of a sialyltransferase, thereby creating a linkage that contains a cyclic succinimide group. However, as already discussed, succinimide based linkages may undergo (undesired) hydrolytic ring opening during storage.

It follows from the above that it is preferable to link the half-life extending moiety to FVIII in such a way that 1) the glycan residue of the glycoprotein is preserved in intact form, and 2) no heterogeneity is present in the linker part between the intact glycosyl residue and the half-life extending moiety.

There is thus a need in the art for methods of conjugating two compounds, such as a half-life extending moiety (such as HEP) to FVIII, wherein the compounds are linked such that a stable and isomer free conjugate is obtained.

In one embodiment a stable and isomer free linker is provided for use in sialic acid based conjugation of HEP to FVIII wherein the HEP polymer may be attached to the sialic acid at positions appropriate for derivatization. Appropriate sites are known to the skilled person, or can be deduced from WO03031464 (which is hereby incorporated by reference in its entirety), where, for example, PEG polymers are attached to sialic acid cytidine monophosphate in multiple ways.

In one embodiment, a stable and isomer free linker is provided for use in glycyl sialic acid cytidine monophosphate (GSC) based conjugation of two compounds, such as a half-life extending moiety conjugated to FVIII, such as HEP conjugated to FVIII.

The GSC starting material used in the current invention can be synthesised chemically (Dufner, G. Eur. J. Org. Chem. 2000, 1467-1482) or, more preferably, it can be obtained by chemoenzymatic routes as described in WO07056191. The GSC structure and carbon atom numbering of the sialic acid part is shown below (Chem.1):

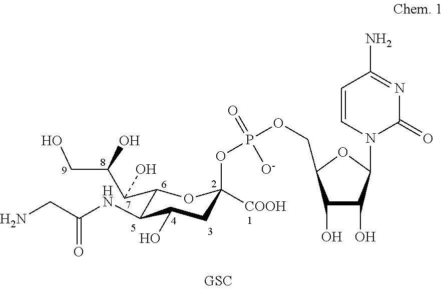

In certain embodiments, the C4 and C5 position of the sialic acid pyranose ring, as well as the C7, C8 and C9 position of the side chain can serve as points of derivatization. Derivatization preferably involves the existing hetero atoms of the sialic acid, such as the hydroxyl or amine group of the glycyl amino (NH₂—CH₂—C(O)NH—) part, but functional group conversion to render appropriate attachment points on the sialic acid is also a possibility.

In one embodiment, the 9-hydroxy group of the sialic acid N-acetylneuraminic acid may be converted to an amino group by methods known in the art (Eur. J. Biochem 168, 594-602 (1987)). The resulting 9-deoxy-amino N-acetylneuraminic acid cytidine monophosphate as shown below (Chem. 5) is an activated sialic acid derivative that can serve as an alternative to GSC.

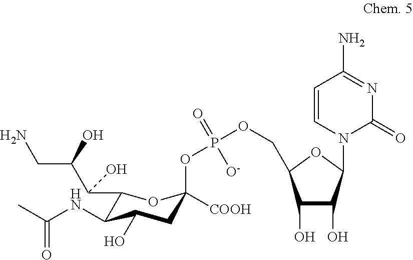

In another embodiment, non-amine containing sialic acids such as 2-keto-3-deoxy-nonic acid, also known as KDN may also be converted to 9-amino derivatized sialic acids following the same scheme (Chem.6):

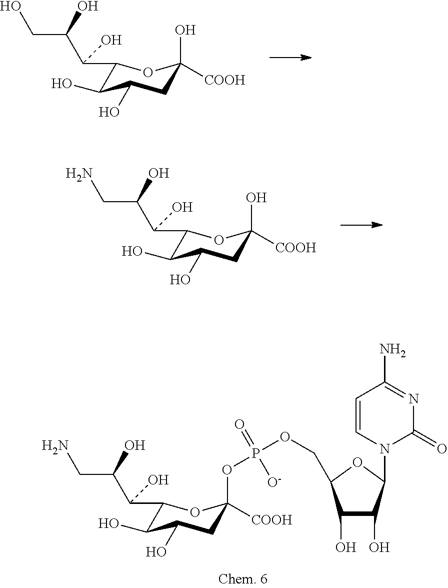

A similar scheme can be used for the shorter C8-sugar analogues belonging to the sialic acid family. Thus shorter versions of sialic acids such as 2-keto-3-deoxyoctonate, also known as KDO may be converted to the 8-deoxy-8-amino-2-keto-3-deoxyoctonate cytidine monophosphate, and used as an alternative to GSC.

As yet another embodiment, neuraminic acid cytidine monophosphate (Chem. 7) may be used in the invention. This material can be prepared, for example, as described in Eur. J. Org. Chem. 2000, 1467-1482.

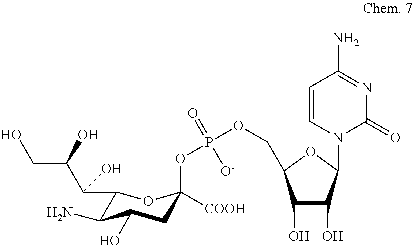

In one embodiment, conjugates according to the present invention comprise a linker comprising the following structure:

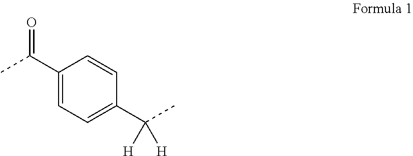

hereinafter also referred to as sublinker or sublinkage—that connects a HEP-amine and GSC in one of the following ways:

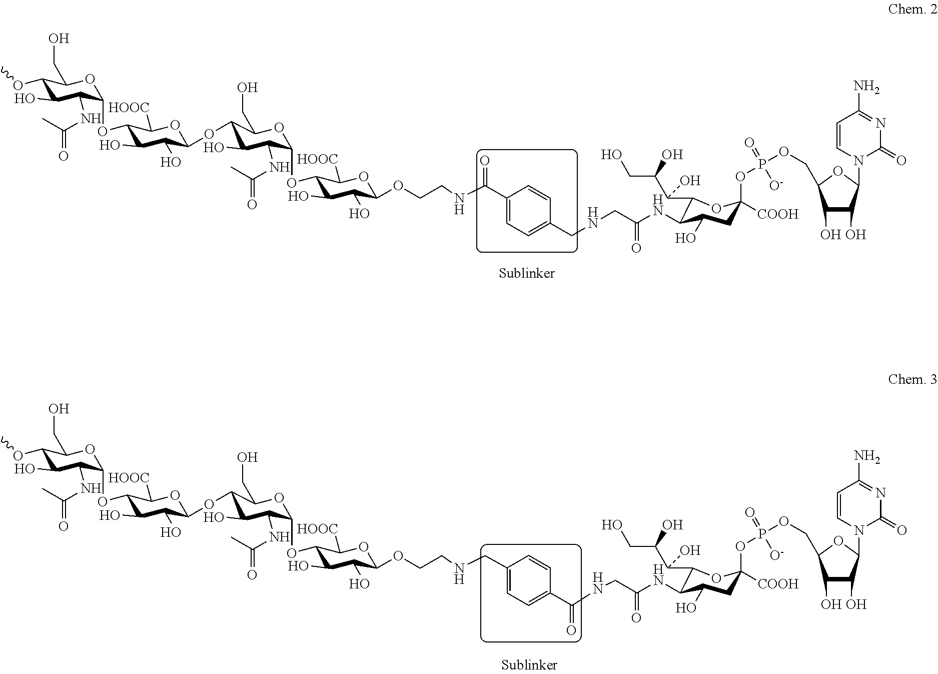

The highlighted 4-methylbenzoyl sublinker thus makes up part of the full linking structure linking the half-life extending moiety to a target protein. The sublinker is a stable structure compared to alternatives, such as succinimide based linkers because, as already discussed, the latter type of cyclic linkage has a tendency to undergo hydrolytic ring opening during storage. One advantage associated with conjugates described herein is that a homogenous composition is obtained, i.e. that the tendency of isomer formation due to linker structure and stability is significantly reduced. Another advantage is that the conjugates prepared according to the described methods can be produced in a simple process, preferably a one-step process.

The 4-methylbenzoyl sublinkage as used in the present invention between HEP and GSC is not able to form stereo- or regio isomers. Processes for preparation of functional HEP polymers are described in US 20100036001 disclosing for example lists aldehyde-, amine- and maleimide functionalized HEP reagents. A range of other functionally modified HEP derivatives are available using similar chemistry. HEP polymers used herein are initially produced with a primary amine handle at the reducing terminal according to methods described in US20100036001.

HEP reagents modified with a benzaldehyde functionality can be kept stable for extended time periods when stored frozen (−80° C.) in dry form.

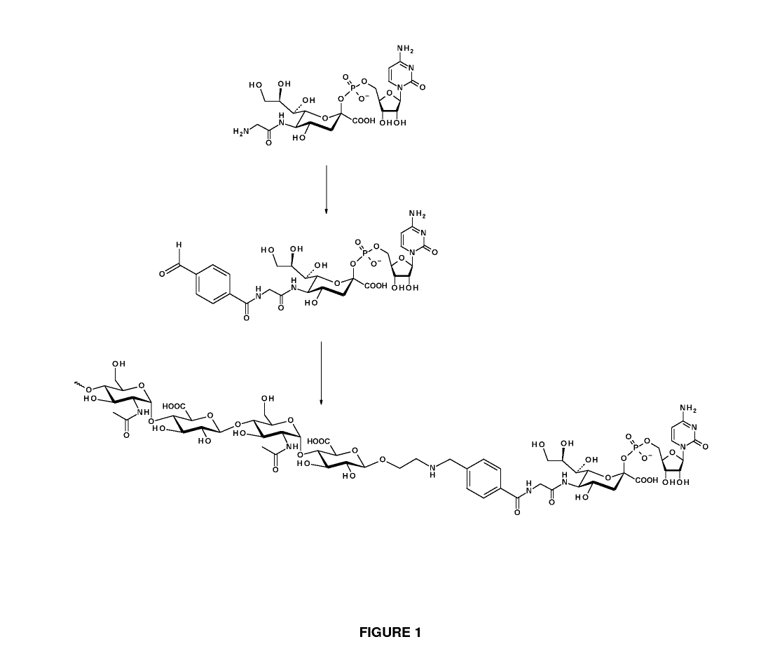

Alternatively, a benzaldehyde moiety can be attached to the GSC compound, thereby resulting in a GSC-benzaldehyde compound suitable for conjugation to an amine functionalized half-life extending moiety. This route of synthesis is depicted in FIG. 1.

For example, GSC can be reacted under pH neutral conditions with N-succinimidyl 4-formylbenzoate to provide a GSC compound that contains a reactive aldehyde group, see for example WO11101267. The aldehyde derivatized GSC compound (GSC-benzaldehyde) can then be reacted with HEP-amine and reducing agent to form a HEP-GSC reagent.

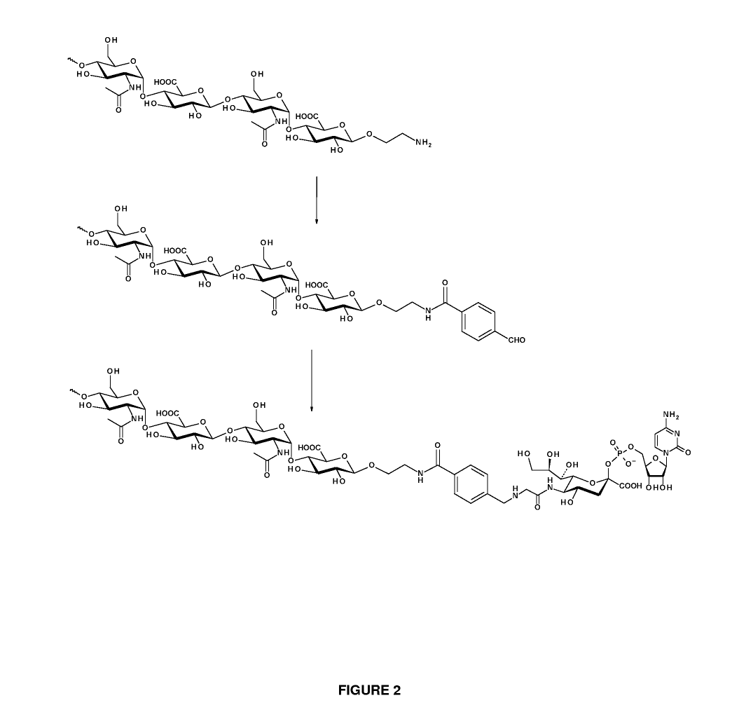

The above mentioned reaction may be reversed, so that the HEP-amine is first reacted with N-succinimidyl 4-formylbenzoate to form an aldehyde derivatized HEP-polymer, which subsequently is reacted directly with GSC in the presence of a reducing agent. In practice this eliminates the tedious chromatographic handling of GSC-CHO. This route of synthesis is depicted in FIG. 2.

Thus, in one embodiment of the present invention HEP-benzaldehyde is coupled to GSC by reductive amination.

Reductive amination is a two-step reaction which proceeds as follows: Initially an imine (also known as Schiff-base) is formed between the aldehyde component and the amine component (in the present embodiment the glycyl amino group of GSC). The imine is then reduced to an amine in the second step. The reducing agent is chosen so that it selectively reduces the formed imine to an amine derivative.

A number of suitable reducing reagents are available to the skilled person. Non-limiting examples include sodium cyanoborohydride (NaBH₃CN), sodium borohydride (NaBH₄), pyridin boran complex (BH₃:Py), dimethylsulfide boran complex (Me₂S:BH₃) and picoline boran complex.

Although reductive amination to the reducing end of carbohydrates (for example to the reducing termini of HEP polymers) is possible, it has generally been described as a slow and inefficient reaction (J C. Gildersleeve, Bioconjug Chem. 2008 July; 19(7): 1485-1490). Side reactions, such as the Amadori reaction, where the initially formed imine rearrange to a keto amine are also possible, and will lead to heterogenecity which as previously discussed is undesirable in the present context.

Aromatic aldehydes such as benzaldehydes derivatives are not able to form such rearrangement reactions as the imine is unable to enolize and also lack the required neighbouring hydroxy group typically found in carbohydrate derived imines. Aromatic aldehydes such as benzaldehydes derivatives are therefore particular useful in reductive amination reactions for generating isomer free HEP-GSC reagent.

A surplus of GSC and reducing reagent is optionally used in order to drive reductive amination chemistry fast to completion. When the reaction is completed, the excess (non-reacted) GSC reagent and other small molecular components such as excess reducing reagent can subsequently be removed by for example dialysis, tangential flow filtration or size exclusion chromatography.

Both the natural substrate for sialyltransferases, Sia-CMP, and the GSC derivatives are multifunctional, charged and highly hydrophilic compounds, which can be difficult to modify and isolate using standard chromatographic methods. In addition, they are not stable in solution for extended time periods, especially if pH is below 6.0. At such low pH, the CMP activation group necessary for substrate transfer is lost due to acid catalyzed phosphate diester hydrolysis. Selective modification and isolation of Sia-CMP derivatives thus require careful control of pH, as well as fast and efficient isolation methods, in order to avoid CMP-hydrolysis.

Large half-life extending moieties may be conjugated to GSC using reductive amination chemistry. Arylaldehydes, such as benzaldehyde modified HEP polymers have been found optimal for this type of modification, as they can efficiently react with GSC under reductive amination conditions.

As GSC may undergo hydrolysis in acid media, it is important to maintain a near neutral or slightly basic environment during the coupling to HEP-benzaldehydes. HEP polymers and GSC are both highly water soluble and aqueous buffer systems are therefore preferable for maintaining pH at a near neutral level. A number of both organic and inorganic buffers may be used, however, the buffer components should preferably not be reactive under reductive amination conditions. This exclude for instance organic buffer systems containing primary and—to lesser extend—secondary amino groups. The skilled person will know which buffers are suitable and which are not. Some examples of suitable buffers are shown in Table 1 below:

TABLE 1
Buffers

| Common | pKa at | Buffer | |
|---|---|---|---|
| Name | 25° C. | Range | Full Compound Name |
| Bicine | 8.35 | 7.6-9.0 | N,N-bis(2-hydroxyethyl)glycine |
| Hepes | 7.48 | 6.8-8.2 | 4-2-hydroxyethyl-1-piperazineethanesulfonic |
| | | | acid |
| TES | 7.40 | 6.8-8.2 | 2-{[tris(hydroxymethyl)methyl]amino} |
| | | | ethanesulfonic acid |
| MOPS | 7.20 | 6.5-7.9 | 3-(N-morpholino)propanesulfonic acid |
| PIPES | 6.76 | 6.1-7.5 | Piperazine-N,N′-bis(2-ethanesulfonic acid) |
| MES | 6.15 | 5.5-6.7 | 2-(N-morpholino)ethanesulfonic acid |

By applying this method, GSC reagents modified with half-life extending moieties (such as HEP), having isomer free stable linkages can efficiently be prepared, and isolated in a simple process that minimize the chance for hydrolysis of the CMP activation group.

By reacting either of said compounds with each other a HEP-GSC conjugate comprising a 4-methylbenzoyl sublinker moiety may be created.

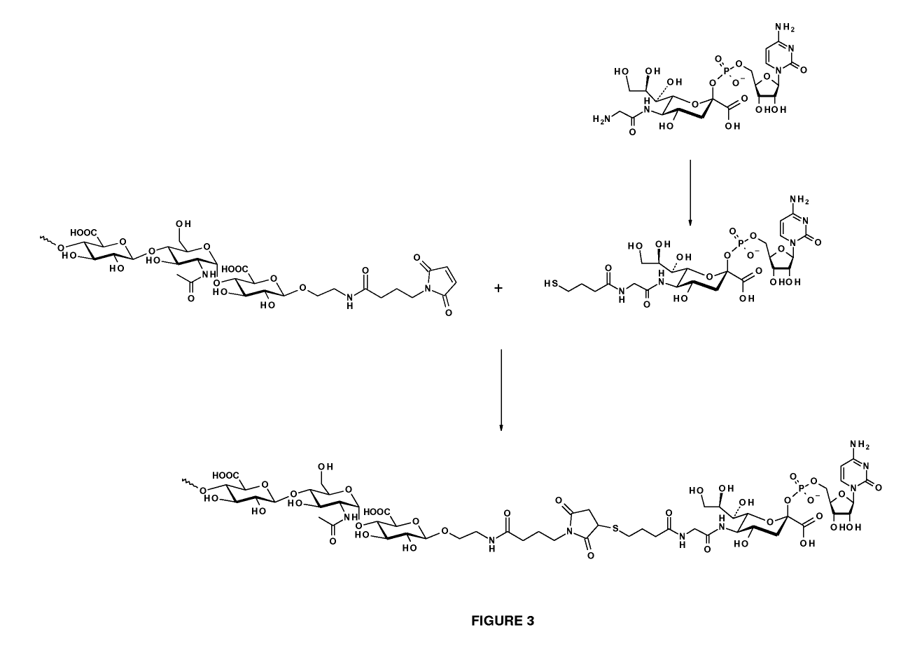

GSC may also be reacted with thiobutyrolactone, thereby creating a thiol modified GSC molecule (GSC-SH). Such reagents may be reacted with maleimide functionalized HEP polymers to form HEP-GSC reagents. This synthesis route is depicted in FIG. 3. The resulting product has a linkage structure (Chem.8) comprising succinimide:

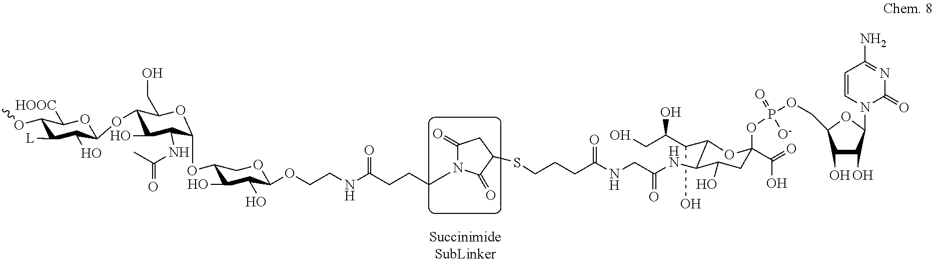

However, succinimide based (sub)linkages may undergo hydrolytic ring opening inter alia when the modified GSC reagent is stored in aqueous solution for extended time periods and while the linkage may remain intact, the ring opening reaction will add undesirable heterogeneity in form of regio- and stereo-isomers.

### Methods of Glycoconjugation

Conjugation of a HEP-GSC conjugate with FVIII may be carried out via a glycan present on FVIII. This form of conjugation is also referred to as glyco-conjugation.

In contrast to conjugation methods based on cysteine alkylations, lysine acylations and similar conjugations involving amino acids in the protein backbone, conjugation via glycans is an appealing way of attaching larger structures such as polymers of protein/peptide fragments to bioactive proteins with less disturbance of bioactivity. This is because glycans being highly hydrophilic generally tend to be oriented away from the protein surface and out in solution, leaving the binding surfaces that are important for the proteins activity free.

The glycan may be naturally occurring or it may be inserted via e.g. insertion of an N-linked glycan using methods well known in the art.

Methods for glycoconjugation of HEP polymers include galactose oxidase based conjugation (WO2005014035) and periodate based conjugation (WO08025856). Methods based on sialyltransferase have over the years proven to be mild and highly selective for modifying N-glycans or O-glcyans on blood coagulation factors.

In contrast to chemical conjugation methods based on cysteine alkylations, lysine acylations and similar conjugations involving amino acids in the protein backbone, conjugation via glycans is an appealing way of attaching larger structures such as polymers of protein/peptide fragments to bioactive proteins with less disturbance of bioactivity. This is because glycans being highly hydrophilic generally tend to point away from the protein surface and out in solution, leaving binding sites important for protein activity free. The glycan may be naturally occurring or it may be inserted via e.g. insertion of an N-linked glycan using genetic engineering methods well known in the art.

GSC is a sialic acid derivative that can be transferred to glycoproteins, such as FVIII, by the use of sialyltransferases. It can be selectively modified with substituents, such as PEG or HEP, on the glycyl amino group and still be enzymatically transferred to glycoproteins by use of sialyltransferases. GSC can be efficiently prepared by an enzymatic process in large scale (WO07056191).

Terminal sialic acids on glycoproteins can be removed by sialidase treatment to provide asialo glycoproteins. Asialo glycoproteins and GSC modified with the half-life extending moiety together can act as substrates for sialyltransferases. The product of the reaction is a glycoprotein conjugate having the half-life extending moiety linked via an intact glycosyl linking group on the glycan.

### Sialyltransferases

Sialyltransferases are a class of glycosyltransferases that transfer sialic acid from naturally activated sialic acid (Sia)—CMP (cytidine monophosphate) compounds to galactosyl-moieties on e.g. proteins. Many sialyltransferases (ST3GaI-III, ST3GaI-I, ST6GaINAc-I) are capable of transfer of Sia-CMP derivatives that has been modified on the C5 acetamido group (WO03031464). A non-limited, list of relevant sialyltransferases, that can be used with the current invention are disclosed in WO2006094810.

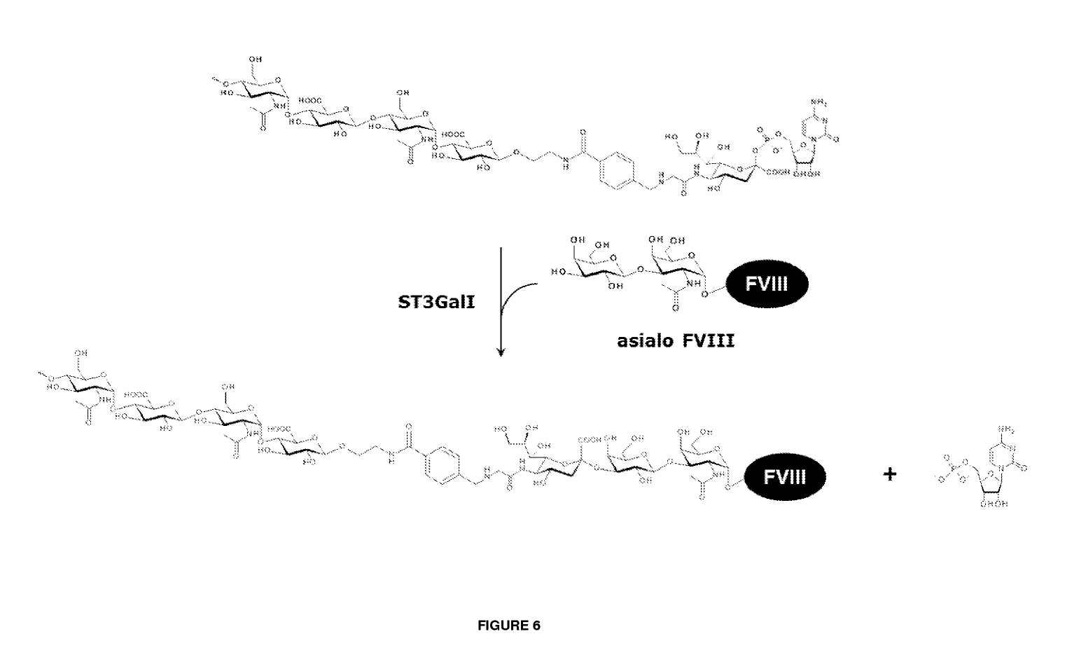

Terminal sialic acids on FVIII can be removed by sialidase treatment to provide asialo FVIII. Asialo FVIII and GSC, modified with the half-life extending moiety, can act as substrates for sialyltransferases. The product of the reaction is a FVIII conjugate having the half-life extending moiety linked via an intact glycosyl linking group—in this case an intact sialic acid linker group. A reaction scheme where an asialo FVIII glycoprotein is reacted with HEP-GSC, in the presence of sialyltransferase, is shown in FIG. 6.

In the examples, sialyltransferase ST3GaI-I is used to generate a conjugate where HEP is attached to an O-glycan on FVIII. If sialyltransferase ST3GaI-III had been chosen, a conjugate having HEP attached to the N-glycans would have been made.

### Properties of HEP-FVIII Conjugates

In some embodiments, the HEP-FVIII conjugates described herein have various advantageous properties. For example, the conjugate may show one or more of the following (non-limiting) advantages compared to a suitable FVIII control molecule:
improved in vivo circulatory half time,
improved mean residence time in vivo
improved biodegradability in vivo,
improved bleeding time and blood loss in a tail vein transection (TVT) model in FVIII knock-out mice,
improved inter-assay variability in various aPTT-based assays.

The conjugate may show an improvement in any biological activity of FVIII as described herein and this may be measured using any assay or method as described herein, such as the methods described below in relation to the activity of FVIII (for example, as described in the Examples section).

Advantages may be seen when a conjugate of the invention is compared to a suitable control FVIII molecule. The control molecule may be, for example, an unconjugated FVIII polypeptide or a conjugated FVIII polypeptide. The conjugated control may be a FVIII polypeptide conjugated to a water soluble polymer, or a FVIII polypeptide chemically linked to a protein. A conjugated VIII control may be a FVIII polypeptide that is conjugated to a chemical moiety (being protein or water soluble polymer) of a similar size as the HEP molecule in the conjugate of interest. The water-soluble polymer can for example be PEG, branched PEG, or Hydroxy Alkyl Starch (HAS), such as Hydroxy Ethyl Starch (HES),

The FVIII polypeptide in the control FVIII molecule is preferably the same FVIII polypeptide that is present in the conjugate of interest. For example, the control FVIII molecule may have the same amino acid sequence as the FVIII polypeptide in the conjugate of interest. The control FVIII may have the same glycosylation pattern as the FVIII polypeptide in the conjugate of interest.

In some embodiments, conjugates as described herein have an improvement in circulatory half-life, or in mean residence time when compared to a suitable control.

In some embodiments, HEP-FVIII conjugates as described herein have a modified circulatory half-life compared to the wild type FVIII molecule, preferably an increased circulatory half-life. Circulatory half-life is preferably increased at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, more preferably at least 125%, more preferably at least 150%, more preferably at least 175%, more preferably at least 200%, and most preferably at least 250% or 300%. Even more preferably, such molecules have a circulatory half-life that is increased at least 400%, 500%, 600%, or even 700%.

Where the activity being compared is a biological activity of FVIII, such as clotting activity or activity in a chromogenic assay, the control can be a suitable FVIII polypeptide conjugated to a water soluble polymer of comparable size to the HEP conjugate of the current invention.

The conjugate may not retain the level of biological activity seen in FVIII that is not modified by the addition of HEP. Preferably, the conjugate retains as much of the biological activity of unconjugated FVIII as possible. For example, the conjugate may retain at least 15%, at least 20%, at least 25%, at least 30%, at least 35%, at least 40%, at least 45%, at least 50%, at least 60%, at least 70%, at least 80% or at least 90% of the biological activity of an unconjugated FVIII control. As discussed above, the control may be a FVIII molecule having the same amino acid sequence as the FVIII polypeptide in the conjugate, but lacking HEP. The conjugate may, however, show an improvement in biological activity when compared to a suitable control. The biological activity here may be any biological activity of FVIII as described herein such as clotting activity or activity in a chromogenic assay.

An improved biological activity when compared to a suitable control as described herein may be any measurable or statistically significant increase in a biological activity. The biological activity may be any biological activity of FVIII as described herein, such as clotting activity, activity in a chromogenic assay, reduction of bleeding time and blood loss. The increase may be, for example, an increase of at least 5%, at least 10%, at least 15%, at least 20%, at least 25%, at least 30%, at least 35%, at least 40%, at least 45%, at least 50%, at least 55%, at least 60%, at least 70% or more in the relevant biological activity when compared to the same activity in a suitable control.

An advantage of the conjugates as described herein is that HEP polymers are enzymatically biodegradable. The conjugates are therefore preferably enzymatically degradable in vivo.

In some embodiments, the conjugates comprising a HEP polymer linked to FVIII reduces or does not cause significant inter-assay variability in when using different aPTT-based clotting assays.

### Compositions

Described are also compositions that comprise HEP-FVIII conjugates as described herein. In some embodiments, the pharmaceutical composition comprises one or more conjugates formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferred pharmaceutically acceptable carriers comprise aqueous carriers or diluents.

The pharmaceutical compositions are primarily intended for parenteral administration for prophylactic and/or therapeutic treatment. Preferably, the pharmaceutical compositions are administered parenterally, i.e., intravenously, subcutaneously, or intramuscularly, or it may be administered by continuous or pulsatile infusion. The compositions for parenteral administration comprise the described HEP-FVIII FVIII conjugate in combination with, preferably dissolved in, a pharmaceutically acceptable carrier, preferably an aqueous carrier. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The concentration of HEP-FVIII conjugate in these formulations can vary widely, i.e., from less than about 0.5% by weight, usually at or at least about 1% by weight to as much as 15 or 20% by weight and will be selected primarily by fluid volumes, viscosities, etc., in accordance with the particular mode of administration selected. Actual methods for preparing parenterally administrable compositions will be known or apparent to those skilled in the art and are described in more detail in, for example, Remington's Pharmaceutical Sciences, 18th ed., Mack Publishing Company, Easton, Pa. (1990).

The composition can be formulated as a solution, microemulsion, liposome, or other ordered structure suitable to high drug concentration.

Compositions may be formulated in dosage unit form for ease of administration and uniformity of dosage. Dosage unit form as used herein refers to physically discrete units suited as unitary dosages for the subjects to be treated; each unit containing a predetermined quantity of conjugate calculated to produce the desired therapeutic effect. The specification for the dosage unit forms of the presently claimed and disclosed invention(s) are dictated by and directly dependent on (a) the unique characteristics of the HEP conjugate and the particular therapeutic effect to be achieved, and (b) the limitations inherent in the art of compounding such a therapeutic compound for the treatment of a selected condition in a subject.

Pharmaceutical compositions as described herein may comprise additional active ingredients in addition to a conjugate as described herein. For example, a pharmaceutical composition may comprise additional therapeutic or prophylactic agents. For example, where a pharmaceutical composition is intended for use in the treatment of a bleeding disorder, it may additionally comprise one or more agents intended to reduce the symptoms of the bleeding disorder. For example, the composition may comprise one or more additional clotting factors. The composition may comprise one or more other components intended to improve the condition of the patient. The composition may be formulated for use in a particular method or for administration by a particular route.

### Uses of the Conjugates

HEP-FVIII conjugates as described herein may be administered to an individual in need thereof in order to deliver FVIII polypeptides to that individual. The individual may be any individual in need of FVIII polypeptides.

The HEP-FVIII conjugates described herein may be used to control bleeding disorders which may be caused by, for example, clotting factor deficiencies (e.g. haemophilia A) or clotting factor inhibitors, or they may be used to control excessive bleeding occurring in subjects with a normally functioning blood clotting cascade (no clotting factor deficiencies or inhibitors against any of the coagulation factors).

The compositions containing the described HEP-FVIII conjugates can be administered for prophylactic and/or therapeutic treatments. In therapeutic applications, compositions are administered to a subject already suffering from a disease, such as any bleeding disorder as described above, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease or injury as well as the weight and general state of the subject. In general, however, the effective delivery amount will range from about 0.05 mg up to about 500 mg of the HEP-FVIII conjugate per day for a 70 kg subject, with dosages of from about 1.0 mg to about 100 mg of the conjugate being delivered per day being more commonly used.

PEGylation has for years been one of the preferred half-life extension technologies for generating long acting drugs, and several PEG-protein conjugates have now reached the market. PEG polymers have a tendency to lower the activity of the protein drug to which it is bound. This typically results in lower drug-receptor affinity or lower binding affinity to the respective drug binding partners in solution. In most cases, the lowering of activity correlate with either PEG size or number of PEG groups attached to the protein drug and attachment of large PEG groups typically leads to considerable higher activity loss than attachment of small PEG groups.

Beside the activity modulating effect of PEG size and PEG numbers, PEG has recently been shown to have strong interference with standard assays used in haemostasis. For example the specific activity of glycoPEGylated FVIII measured in one-stage clotting assays vary depending on the aPTT reagent used (Stennicke, Blood 2013; 121(11):2108-16).

Use of the aPTT one-stage FVIII clotting assay is a standard procedure used for individual optimisation of the dose- and dosing regimens during initiation of treatment and for routine monitoring of FVIII prophylaxis. In general, aPTT assays are conducted at a central laboratory where clotting of blood obtained from the patient is initiated by addition of an aPTT reagent and re-calcification after which time to fibrin clot formation is measured on a coagulation analyser. There are many commercially available formats of this assay.

The assay interfering property of PEG may have significant impact in preclinical development and even more so in clinical application where precise measurement of patients' blood coagulation factors in multi component one-stage clotting assay are required.

In one embodiment, the HEP-FVIII conjugates described herein show improved performance compared to similar PEGylated FVIII conjugates in aPTT assays. In one embodiment, the HEP-FVIII conjugates described herein reduces inter-assay variability in aPTT-based assays compared to inter-assay aPTT variability when assaying similar pegylated FVIII conjugates (PEG-FVIII).

### DEFINITIONS

Unless defined otherwise, all technical and scientific terms used herein generally have the same meaning as commonly understood by a person of ordinary skill in the art.

The term “subject”, as used herein, includes any human patient or non-human vertebrate.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to treating a disease in said human or other vertebrate. The timing and purpose of said treatment may vary from one individual to another, according to the subject's health. Thus, said treatment may be prophylactic, palliative, symptomatic and/or curative.

Mode of administration: Compounds (conjugates) and pharmaceutical compositions comprising HEP-FVIII conjugates as described herein may be administered parenterally, such as e.g. intravenously or extravascularly (such as e.g. intradermally, intramuscularly, subcutaneously, etc). Compounds and pharmaceutical compositions comprising the herein described HEP-FVIII conjugates may be administered prophylactically and/or therapeutically and/or on demand.

Combination treatments/co-administration: Combined administration of two or more active compounds may be achieved in a number of different ways. In one embodiment, the two active compounds may be administered together in a single composition. In another embodiment, the two active compounds may be administered in separate compositions as part of a combined therapy.

The term “coagulopathy” refers to an increased haemorrhagic tendency which may be caused by any qualitative or quantitative deficiency of any pro-coagulative component of the normal coagulation cascade, or any upregulation of fibrinilysis. Such coagulopathies may be congenital and/or acquired and/or iatrogenic and are identified by a person skilled in the art.

Non-limiting examples of congenital hypocoagulopathies include haemophilia A. The clinical severity of haemophilia A is determined by the concentration of functional units of FVIII in the blood and is classified as mild, moderate, or severe. Severe haemophilia is defined by a clotting factor level of <0.01 U/ml corresponding to <1% of the normal level, while moderate and mild patients have levels from 1-5% and >5%, respectively. Haemophilia A with “inhibitors” (that is, allo-antibodies against factor VIII) is a non-limiting examples of a coagulopathy that is partly congenital and partly acquired.

The term “half-life” as used herein in the context of administering a peptide drug to a patient, is defined as the time required for plasma concentration of a drug in a patient to be reduced by one half.

The term “half-life extending moiety” (or “side chain”) is herein understood to refer to one or more chemical groups that can increase in vivo circulation half-life of a number of therapeutic proteins/peptides when conjugated to these proteins/peptides. Examples of half-life extending moieties include: biocompatible fatty acids and derivatives thereof, Hydroxy Alkyl Starch (HAS) e.g. Hydroxy Ethyl Starch (HES), Poly Ethylen Glycol (PEG), heparosan, and any combination thereof.

The term “sialic acid” refers to any member of a family of nine-carbon carboxylated sugars. The most common member of the sialic acid family is N-acetylneuraminic acid (2-keto-5-acetamido-3,5-dideoxy-D-glycero-D-galactononulopyranos-1-onic acid (often abbreviated as Neu5Ac, NeuAc, NeuNAc, or NANA). A second member of the family is N-glycolyl-neuraminic acid (Neu5Gc or NeuGc), in which the N-acetyl group of NeuNAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O-C1-C6 acyl-Neu5Ac like 9-O-lactylNeu5Ac or 9-O-acetyl-Neu5Ac. The synthesis and use of sialic acid compounds in a sialylation procedure is disclosed in international application WO92/16640, published Oct. 1, 1992.

The term “sialic acid derivative” refers to sialic acids as defined above that are modified with one or more chemical moieties. The modifying group may for example be alkyl groups such as methyl groups, azido- and fluoro groups, or functional groups such as amino or thiol groups that can function as handles for attaching other chemical moieties. Examples include 9-deoxy-9-fluoro-Neu5Ac and 9-azido-9-deoxy-Neu5Ac. The term also encompasses sialic acids that lack one of more functional groups such as the carboxyl group or one or more of the hydroxyl groups. Derivatives where the carboxyl group is replaced with a carboxamide group or an ester group are also encompassed by the term. The term also refers to sialic acids where one or more hydroxyl groups have been oxidized to carbonyl groups. Furthermore the term refers to sialic acids that lack the C9 carbon atom or both the C9-C8 carbon chain for example after oxidative treatment with periodate.

Glycyl sialic acid is a sialic acid derivative according to the definition above, where the N-acetyl group of NeuNAc is replaced with a glycyl group also known as an amino acetyl group. Glycyl sialic acid may be represented with the following structure (the carbon atom numbering of the sialic acid part is shown by Chem. 1, above):

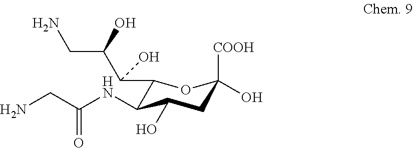

The term “CMP-activated” sialic acid or sialic acid derivatives refer to a sugar nucleotide containing a sialic acid moiety and a cytidine monophosphate (CMP).

In the present description, the term “glycyl sialic acid cytidine monophosphate” is used for describing GSC, and is a synonym for alternative naming of same CMP activated glycyl sialic acid. Alternative naming include CMP-5′-glycyl sialic acid, cytidine-5′-monophospho-N-glycylneuraminic acid, cytidine-5′-monophospho-N-glycyl sialic acid.

The term “intact glycosyl linking group” refers to a linking group that is derived from a glycosyl moiety in which the saccharide monomer interposed between and covalently attached to the polypeptide and the HEP moiety is not degraded, e.g., oxidized, e.g., by sodium metaperiodate during conjugate formation. “Intact glycosyl linking groups” may be derived from a naturally occurring oligosaccharide by addition of glycosyl unites or removal of one or more glycosyl unit from a parent saccharide structure.

The term “asialo glycoprotein” is intended to include glycoproteins wherein one or more terminal sialic acid residues have been removed, e.g., by treatment with a sialidase or by chemical treatment, exposing at least one galactose or N-acetylgalactosamine residue from the underlying “layer” of galactose or N-acetylgalactosamine (“exposed galactose residue”).

The term “glycan” refers to the entire oligosaccharide structure that is covalently linked to a single amino acid residue. Glycans are normally N-linked or O-linked, e.g., glycans are linked to an asparagine residue (N-linked glycosylation) or a serine or threonine residue (O-linked glycosylation). N-linked oligosaccharide chains may be multi-antennary, such as, e.g., bi-, tri, or tetra-antennary and most often contain a core structure of Man3-GIcNAc-GIcNAc-. Both N-glycans and O-glycans are attached to proteins by the cells producing the protein. The cellular N-glycosylation machinery recognizes and glycosylates N-glycosylation consensus motifs (N—X—S/T motifs) in the amino acid chain, as the nascent protein is translocated from the ribosome to the endoplasmic reticulum (Kiely et al. 1976; Glabe et al. 1980). Some glycoproteins, when produced in a human in situ, have a glycan structure with terminal, or “capping”, sialic acid residues, i.e., the terminal sugar of each antenna is N-acetylneuraminic acid linked to galactose via an a2->3 or a2->6 linkage. Other glycoproteins have glycans end-capped with other sugar residues. When produced in other circumstances, however, glycoproteins may contain oligosaccharide chains having different terminal structures on one or more of their antennae, such as, e.g., containing N-glycolylneuraminic acid (Neu5Gc) residues or containing a terminal N-acetylgalactosamine (GaINAc) residue in place of galactose.

Dotted lines in structure formulas denotes open valence bond (i.e. bonds that connect the structures to other chemical moieties).

### Factor VIII

FVIII conjugates/compounds/molecules/polypeptides herein are capable of functioning in the coagulation cascade in a manner that is functionally similar, or equivalent, to wt/endogenous FVIII, inducing the formation of FXa via interaction with FIXa on activated platelets and supporting the formation of a blood clot. As used herein, the terms “Factor VIII polypeptide” or “FVIII polypeptide” encompass, without limitation, wild-type human FVIII and FVIII as well as polypeptides exhibiting substantially the same or improved biological activity relative to wild-type human FVIII. These polypeptides include, without limitation, FVIII or FVIII that has been chemically modified and FVIII or FVIIIa analogues into which specific amino acid sequence alterations have been introduced that modify the bioactivity of the polypeptide unless otherwise indicated. FVIII activity can be assessed in vitro using techniques well known in the art. Clotting assays, FX activation assays (often termed chromogenic assays), thrombin generation assays and whole blood thrombo-elastography assays are examples of such in vitro techniques. FVIII molecules that may be conjugated to heparosan as described herein have FVIII activity that is at least about 10%, at least about 20%, at least about 30%, at least about 40%, at least about 50%, at least about 60%, at least about 70%, at least about 80%, at least about 90%, 100% or even more than 100% of that of native human FVIII, when measured in one or more of these assays.

Endogenous full length FVIII is synthesized as a single-chain precursor molecule. Prior to secretion, the precursor is cleaved into the heavy chain and the light chain. Recombinant B domain-deleted or truncated FVIII can be produced by means of two different strategies. Either the heavy chain without the B-domain (or with a truncated B domain) and the light chain are synthesized individually as two different polypeptide chains (two-chain strategy) or the B domain-deleted or -truncated FVIII is synthesized as a single precursor polypeptide chain (single-chain strategy) that is cleaved by a protease into the heavy and light chains in the same way as the full-length FVIII precursor.

In a B domain-deleted (or -truncated) FVIII precursor polypeptide, produced by the single-chain strategy, the heavy and light chain moieties are often separated by a linker. In order to be able to function in the coagulation cascade, this FVIII linker must comprise a recognition site for the protease that separates the B domain-deleted FVIII precursor polypeptide into the heavy and light chain. To minimize the risk of introducing immunogenic epitopes in the B domain-deleted/truncated FVIII, the sequence of the linker is preferably derived from the FVIII B-domain. In the B domain of full length FVIII, amino acid 1644-1648 constitutes this recognition site. The thrombin cleavage site leading to removal of the linker on activation of B domain-deleted FVIII is located in the heavy chain. Thus, the size and amino acid sequence of the B domain linker is unlikely to influence its removal from the remaining FVIII molecule by thrombin activation. Deletion/truncation of the B domain is an advantage for production of FVIII. Nevertheless, parts of the B domain can be included in the linker without reducing the productivity. The negative effect of the B domain on productivity has not been attributed to any specific size or sequence of the B domain.

The term “FVIII” as used herein, is intended to designate any FVIII molecule having FVIII activity, including wt FVIII, B domain deleted/truncated FVIII molecules, variants of FVIII exhibiting substantially the same or improved biological activity relative to wt FVIII and FVIII-related polypeptides, in which one or more of the amino acids of the parent peptide have been chemically modified, e.g. by protein:protein fusion, alkylation, PEGylation, HESylation, PASylation, PSAylation, acylation, ester formation or amide formation.

The sequence of wild-type human coagulation Factor VIII is listed below (SEQ ID NO: 1: wt human FVIII (Ser750 residue shown in bold and underline)):

ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
TRQKQFNATTIPENDIEKTDPWFAHRTPMPKIQNVSSSDLLMLLRQSPTP
HGLSLSDLQEAKYETFSDDPSPGAIDSNNSLSEMTHFRPQLHHSGDMVFT
PESGLQLRLNEKLGTTAATELKKLDFKVSSTSNNLISTIPSDNLAAGTDN
TSSLGPPSMPVHYDSQLDTTLFGKKSSPLTESGGPLSLSEENNDSKLLES
GLMNSQESSWGKNVSSTESGRLFKGKRAHGPALLTKDNALFKVSISLLKT
NKTSNNSATNRKTHIDGPSLLIENSPSVWQNILESDTEFKKVTPLIHDRM
LMDKNATALRLNHMSNKTTSSKNMEMVQQKKEGPIPPDAQNPDMSFFKML
FLPESARWIQRTHGKNSLNSGQGPSPKQLVSLGPEKSVEGQNFLSEKNKV
VVGKGEFTKDVGLKEMVFPSSRNLFLTNLDNLHENNTHNQEKKIQEEIEK
KETLIQENVVLPQIHTVTGTKNFMKNLFLLSTRQNVEGSYDGAYAPVLQD
FRSLNDSTNRTKKHTAHFSKKGEEENLEGLGNQTKQIVEKYACTTRISPN
TSQQNFVTQRSKRALKQFRLPLEETELEKRIIVDDTSTQWSKNMKHLTPS
TLTQIDYNEKEKGAITQSPLSDCLTRSHSIPQANRSPLPIAKVSSFPSIR
PIYLTRVLFQDNSSHLPAASYRKKDSGVQESSHFLQGAKKNNLSLAILTL
EMTGDQREVGSLGTSATNSVTYKKVENTVLPKPDLPKTSGKVELLPKVHI
YQKDLFPTETSNGSPGHLDLVEGSLLQGTEGAIKWNEANRPGKVPFLRVA
TESSAKTPSKLLDPLAWDNHYGTQIPKEEWKSQEKSPEKTAFKKKDTILS
LNACESNHAIAAINEGQNKPEIEVTWAKQGRTERLCSQNPPVLKRHQREI
TRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRG
ELNEHLGLLGPYIRAEVEDNIMVTFRNQASRPYSFYSSLISYEEDQRQGA
EPRKNFVKPNETKTYFWKVQHHMAPTKDEFDCKAWAYFSDVDLEKDVHSG
LIGPLLVCHTNTLNPAHGRQVTVQEFALFFTIFDETKSWYFTENMERNCR
APCNIQMEDPTFKENYRFHAINGYIMDTLPGLVMAQDQRIRWYLLSMGSN
ENIHSIHFSGHVFTVRKKEEYKMALYNLYPGVFETVEMLPSKAGIWRVEC
LIGEHLHAGMSTLFLVYSNKCQTPLGMASGHIRDFQITASGQYGQWAPKL
ARLHYSGSINAWSTKEPFSWIKVDLLAPMIIHGIKTQGARQKFSSLYISQ
FIIMYSLDGKKWQTYRGNSTGTLMVFFGNVDSSGIKHNIFNPPIIARYIR
LHPTHYSIRSTLRMELMGCDLNSCSMPLGMESKAISDAQITASSYFTNMF
ATWSPSKARLHLQGRSNAWRPQVNNPKEWLQVDFQKTMKVTGVTTQGVKS
LLTSMYVKEFLISSSQDGHQWTLFFQNGKVKVFQGNQDSFTPVVNSLDPP
LLTRYLRIHPQSWVHQIALRMEVLGCEAQDLY

FVIII may be plasma-derived or recombinantly produced using well known methods of production and purification. The degree and location of glycosylation and other post-translation modifications may vary depending on the chosen host cell and its growth conditions.

Host cells for producing recombinant proteins are preferably of mammalian origin in order to ensure that the molecule is properly processed during folding and post-translational modification, e.g. O and N-glycosylation and sulfatation. Suitable host cells include, without limitation, Chinese Hamster Ovary (CHO), baby hamster kidney (BHK), and HEK293 cell lines.

The B domain in FVIII spans amino acid residues 741-1648 of SEQ ID NO: 1. The B domain is cleaved at several different sites, generating large heterogeneity in circulating plasma FVIII molecules. The exact function of the heavily glycosylated B domain is unknown. What is known is that the B domain is dispensable for FVIII activity in the coagulation cascade. Recombinant FVIII is thus frequently produced in a form of B domain-deleted/truncated variants.

In one embodiment, the FVIII conjugated to HEP is a B-domain truncated FVIII molecule. In one embodiment, the FVIII conjugated to HEP is conjugated via a Cys residue. In one embodiment, the FVIII conjugated to HEP is conjugated via a FVIII glycan; in one embodiment hereof, the glycan is a N-glycan; in an alternative embodiment, the glycan is an 0-glycan. In one embodiment, the FVIII is conjugated to HEP via an O-glycan present on a serine amino acid residue corresponding to Ser750 of SEQ ID NO:1.

A FVIII molecule herein may e.g. be produced by an expression vector encoding a FVIII molecule comprising a 21 amino acid residue L (linker) sequence with the following sequence: SEQ ID NO: 2: SFSQNSRHP [symbol] QNPPVLKRHQR (the 0-glycan is attached to the underlined S).

Alternative preferred B domain linker sequences in the FVIII molecules herein may lack one or more of the amino acid residues set forth in SEQ ID NO: 2. For example, the C-terminal R in SEQ ID NO: 2 may be deleted resulting in a 20 amino acid linker sequence, SFSQNSRHP [symbol] QNPPVLKRHQ (SEQ ID NO: 3). Alternatively, the N-terminal S in SEQ ID NO: 2 may be deleted resulting in the following amino acid linker sequence: FSQNSRHP [symbol] QNPPVLKRHQR (SEQ ID NO: 4).

In one embodiment, the FVIII conjugated to HEP is a B-domain truncated FVIII molecule wherein amino acid residues 1-740 of SEQ ID NO:1 (FVIII heavy chain) and amino acid residues 1649-2332 of SEQ ID NO:1 are linked by means of an amino acid linker sequence, L:

HC (1-740)-L-LC(1649-2332)

wherein L is derived from amino acid residues 741-1648 of SEQ ID NO: 1 (FVIII B-domain) by deletion/truncation.

In one embodiment, the linker sequence, L has the sequence of SEQ ID NO:2. In another embodiment, the linker sequence, L has the sequence of SEQ ID NO:3. In yet another embodiment, the linker sequence, L has the sequence of SEQ ID NO:4.

In one embodiment, the FVIII molecule conjugated to HEP is turocotoc alfa (N8) (as described, for example, by Thim et al., Haemophilia (2010), 16, 349-359).

Preferred FVIII conjugates herein are B domain deleted/truncated variants comprising an O-glycan attached to the Ser 750 residue of SEQ ID NO: 1 (shown in bold and underlined) conjugated to a heparosan polymer via the Ser 750 0-glycan. (The Ser at residue in SEQ ID NOS: 2, 3, and 4 is similarly shown in bold and underlined.).

In different embodiments, the FVIII conjugated to HEP is a B-domain truncated FVIII molecule wherein amino acid residues 1-740 of SEQ ID NO:1 (FVIII heavy chain) and amino acid residues 1649-2332 of SEQ ID NO:1 is linked by means of an amino acid linker sequence, L:

HC (1-740)-L-LC(1649-2332)

wherein L is derived from amino acid residues 741-1648 of SEQ ID NO: 1 (FVIII B-domain) by deletion/truncation, and wherein HEP is conjugated to the FVIII molecule via a glycan attached to Ser 750 of SEQ ID NO: 1, SEQ. ID NO: 2, SEQ ID NO: 3, or SEQ ID NO: 4.

In preferred embodiments of the above, the HEP wherein the molecular weight of the heparosan polymer is 35-45 kDa.

### Further Embodiments

1. A FVIII conjugate comprising a heparosan polymer (HEP), and a linking moiety, wherein the linking moiety between FVIII and HEP comprises X as follows:
   [heparosan polymer]-[X]-[FVIII]
      wherein X comprises a sialic acid glycosyl group connected to the structure according to Formula 1 below:

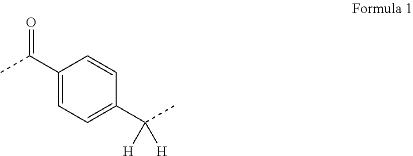

2. The conjugate according to the invention, wherein the sialic acid glycosyl group is glycyl sialic acid according to Formula 3 below:

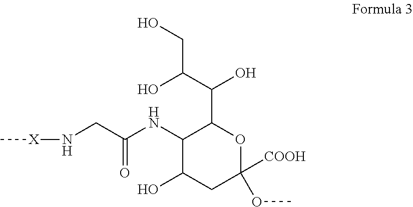

3. The conjugate according to the invention wherein [[heparosan polymer]-[X]] comprises the structure shown in Formula 4 below:

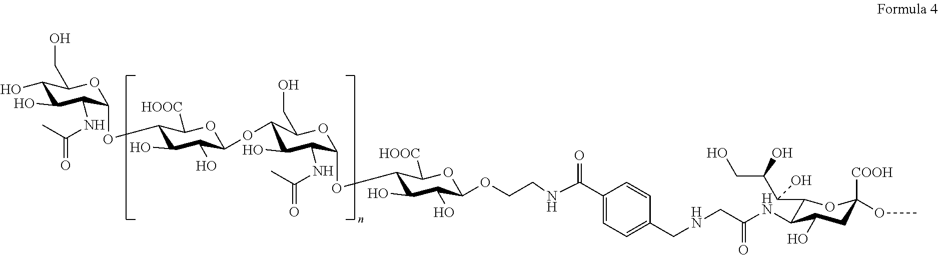

wherein n is an integer from 5 to 450.
4. A conjugate according to the invention, wherein the FVIII molecule is a B domain truncated

FVIII molecule, wherein the sequence of the B domain is selected from the group consisting of SEQ ID NO 2, SEQ ID NO 3, and SEQ ID NO 4.
5. A conjugate according to the invention, wherein the size of the heparosan polymer is 35-45 kDa. The average size of the heparosan polymer in this embodiment is about 40 kDa.
6. A conjugate according to the invention, wherein the heparosan polymer is conjugated to FVIII via an O-linked glycan in the B domain, wherein FVIII activation results in removal of said heparosan polymer.
7. A conjugate according to the invention, wherein said heparosan polymer is linked to FVIII via an O-linked glycan attached to a Serine residue corresponding to the Ser750 residue in SEQ ID NO 1, and wherein the link between FVIII and heparosan comprises the following structure:

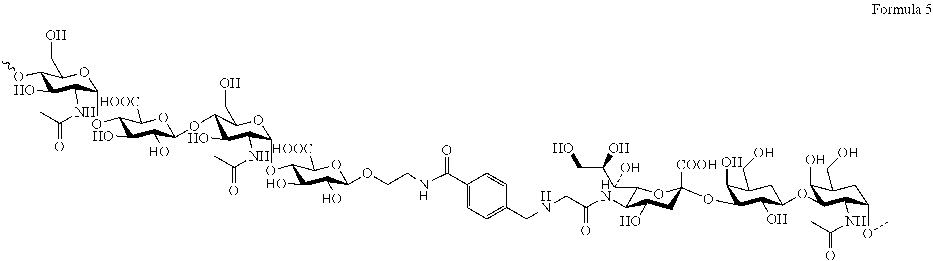

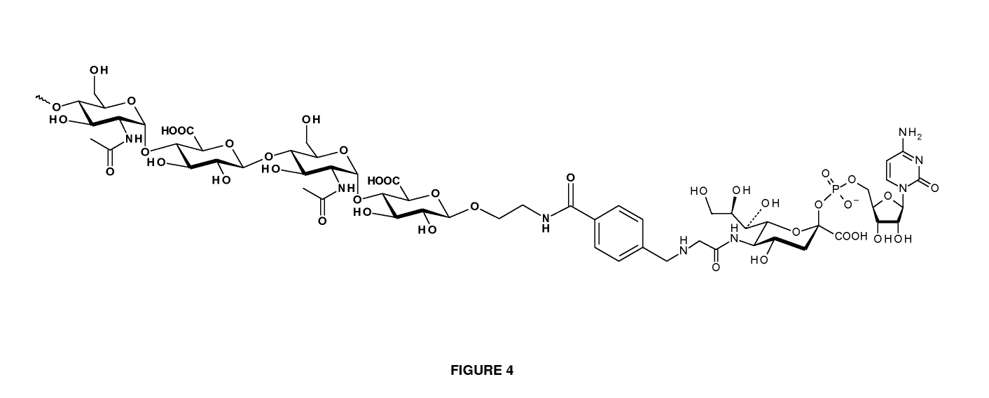

8. A pharmaceutical composition comprising a conjugate according to the invention. The pharmaceutical composition furthermore optionally comprises one or more pharmaceutically acceptable excipients. The formulation can be either lyophilized or in the form a liquid aqueous solution.
9. Use of a conjugate according to the invention for reducing inter-assay variability in an in vitro aPTT-based assay.
10. A conjugate according to the invention for use as a medicament.
11. A conjugate according to the invention for use in treatment of haemophilia.
12. In one embodiment a GSC compound functionalized with a benzaldehyde moiety is provided which is suitable for conjugation with compounds of interest.
13. In one embodiment a benzaldehyde moiety is attached to the GSC compound, thereby resulting in GSC-benzaldehyde compound suitable for conjugation to a half-life extending moiety functionalized with an amine group (cf. FIG. 1).
14. In one embodiment, 4-formylbenzoic acid is chemically coupled to a half-life extending moiety comprising HEP, and subsequently coupled to GSC by reductive amination.
15. In one such embodiment 4-formylbenzoic acid is coupled to HEP (cf. FIG. 2).
16. In a preferred embodiment the invention provides GSC-based conjugation wherein a 4-methylbenzoyl moiety is part of the linking structure (cf. FIG. 4).
17. In one embodiment a first compound comprising a reactive amine is conjugated to a GSC compound functionalized with a benzaldehyde moiety, wherein said amine is reacted with benzaldehyde to yield a (sub)linker between the first compound and GSC which comprises a 4-methylbenzoyl sublinking moiety.
18. In another embodiment a first compound comprising a reactive benzaldehyde is conjugated to the glycyl amine part of a GSC compound, wherein said benzaldehyde is reacted with an amine to yield a (sub)linker between the first compound and GSC which comprises a 4-methylbenzoyl sublinking moiety.
19. In one embodiment the conjugate between the above mentioned first compound and GSC is further conjugated onto a third compound of interest to yield a conjugate where the first compound is linked via a 4-methylbenzoyl sublinking moiety and sialic acid derivative to the third compound of interest.
20. In one embodiment of the present invention a HEP polymer is conjugated to a protein using 4-methylbenzoyl—GSC based conjugation.
21. In one embodiment, a half-life extending moiety comprising an amino group is reacted with 4-formylbenzoic acid and subsequently coupled to the glycyl amino group of GSC by a reductive amination.
22. In one embodiment GSC prepared according to WO07056191 is reacted with a half-life extending moiety comprising a benzaldehyde moiety under reducing conditions.
23. In one embodiment various HEP-benzaldehyde compounds suitable for coupling to GSC are provided.
24. In one embodiment the sublinker between the half-life extending moiety and GSC is not able to form stereo- or regio isomers.
25. In one embodiment the sublinker between the half-life extending moiety and GSC is not able to form stereo- or regio isomers, and therefore has lesser potential for generating immune response in humans.
26. In one embodiment, HEP-GSC is used for preparing an N-glycan and/or an O-glycan HEP FVIII conjugate.
27. In one embodiment, a CMP activated sialic acid derivative used in the present invention is represented by the following structure:

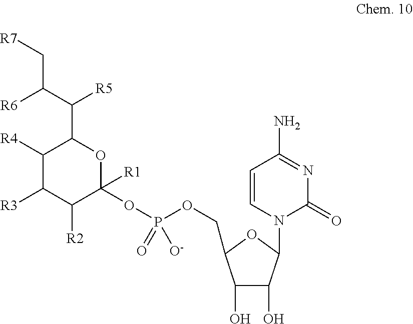

wherein R1 is selected from —COOH, —CONH2, —COOMe, —COOEt, —COOPr and R2. R3, R4, R5, R6 and R7 independently can be selected from —H, —NH₂, —SH, —N3, —OH, —F.

In a preferred embodiment, R1 is —COOH, R2 is —H, R3=R5=R6=R7=-OH and R4 is a glycylamido group (—NHC(O)CH2NH2).

In a preferred embodiment the CMP activated sialic acid is GSC having the following structure:

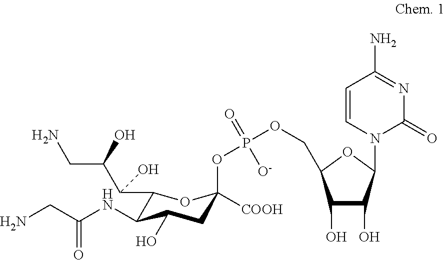

28. In one embodiment, the conjugate according to the invention comprises a FVIII polypeptide, a linking moiety, and a heparosan polymer wherein the linking moiety between the Factor FVIII polypeptide and the heparosan polymer comprises X as follows:
   [heparosan polymer]-[X]-[Factor FVIII polypeptide]
   wherein X comprises a sialic acid derivative connected to a moiety according to Formula 1 below:

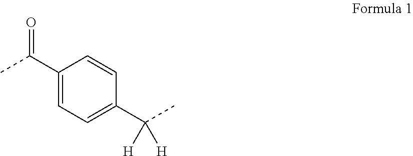

29. In one embodiment, the conjugate according to the invention comprises the sialic acid derivative glycyl sialic acid according to Formula 3 below:

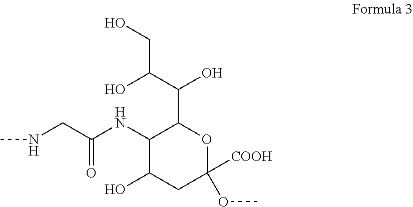

and wherein the moiety of Formula 1 is connected to the terminal —NH handle of Formula 3.
30. In one embodiment, the conjugate according to the invention wherein
   [heparosan polymer]-[X]-
   comprises the structural fragment shown in Formula 4 below:

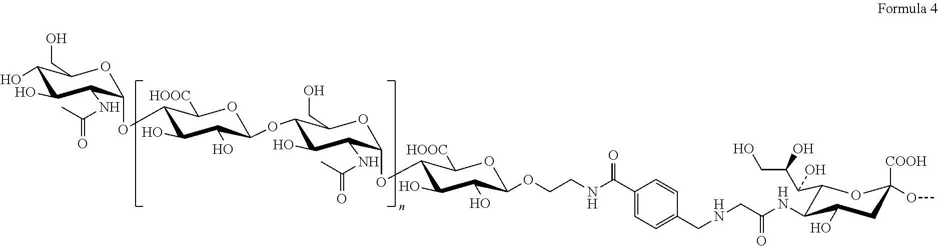

wherein n is an integer from 5 to 450.
31. In one embodiment, the conjugate according to the invention comprises a heparosan polymer having a molecular weight in the range of about 5 to 100 kDa.
32. In one embodiment, the present invention relates to a pharmaceutical composition comprising the conjugate according to the invention.
33. In one embodiment, the present invention relates to use of a heparosan polymer conjugated to a Factor FVIII polypeptide for reducing inter-assay variability in aPTT-based clotting assays (an in vitro or ex in vivo clotting assay).
34. In one embodiment, the present invention relates to use of conjugates according to the invention as a medicament.
35. In one embodiment, the present invention relates to use of conjugates according to the invention for use in the treatment of coagulopathy, such as haemophilia A.
36. In one embodiment, the present invention relates to a method of conjugating a heparosan moiety to a Factor FVIII polypeptide comprising:
   (a) reacting a heparosan moiety with a reactive amine with an activated 4-formylbenzoic acid to yield the compound of Formula 6 below:

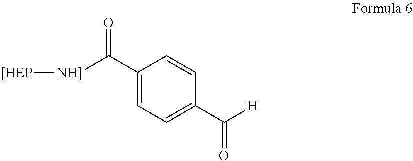

(b) reacting the compound of Formula 6 with a GSC moiety under reducing conditions to yield a compound according to Formula 7 below:

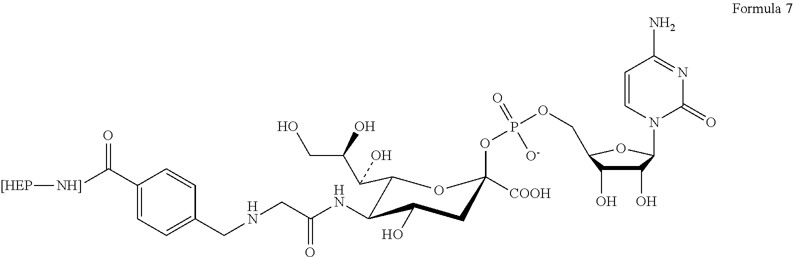

(c) conjugating the compound according to Formula 7 to a Factor FVIII polypeptide.

The present invention furthermore relates to compounds obtained or obtainable by this method.

### EXAMPLES

### Abbreviations Used in Examples

CMP: Cytidine monophosphate
GlcUA: Glucuronic acid
GIcNAc: N-acetylglucosamine
GSC: glycyl sialic acid cytidine monophosphate
GSC-SH: [(4-mercaptobutanoyl)glycyl]sialic acid cytidine monophosphate
HEP: Heparosan
HEP-GSC: GSC-functionalized heparosan polymers
HEP-FVIII Heparosan polymer conjugated to FVIII
HEP-[C]-FVIII Heparosan polymer conjugated to FVIII via a cysteine residue
HEP-[N]-FVIII Heparosan polymer conjugated to FVIII via a N-glycan
HEP-[O]-FVIII Heparosan polymer conjugated to FVIII via a 0-glycan
N8-HEP: Heparosan polymer conjugated via 0-glycan in the B domain to a B domain truncated FVIII.
40k-HEP-[0]-N8 Heparosan polymer having a molecular weight of 40 kDa conjugated via 0-glycan in the B domain to a B domain truncated FVIII.
N8 B-domain truncated FVIII (turoctocog alfa)
Hepes: 2-[4-(2-hydroxyethyl)piperazin-1-yl]ethanesulfonic acid
His: Histidine
PmHS1: Pasteurella multocida Heparosan Synthase I
TCEP: Tris(2-carboxyethyl)phosphine_{—}
UDP: Uridine diphosphate

### Protein Quantification Method

The conjugates of the invention were analysed for purity by HPLC. HPLC was also used to quantify amount of isolated conjugate based on a FVIII reference molecule. A Daiso column (300 Δ; 5 mm; 2.1×250 mm) from FeF Chemicals A/S was used. Column was operated at 40° C. 10 pg sample was injected, and column was eluted with a water (A)—acetonitrile (B) solvent system containing 0.1% trifluoroacetic acid. The gradient program was as follows: 0 min (28% B); 30 min (67% B); 30.5 min (28% B); 40 min (28% B). Depending on conjugate type (O-glycan or cystein conjugation), the non-modified heavy chain or non-modified light chain of the FVIII conjugate were used for quantification relative to a FVIII heavy/light chain standard. For N-glycan modification, the combined area under curve for heavy chain and modified heavy chain were used for quantification relative to FVIII heavy chain standard.

### SDS-PAGE Analysis

SDS PAGE analysis was performed using precast Nupage 7% tris-acetate gel, NuPage tris-acetate SDS running buffer and NuPage LDS sample buffer all from Invitrogen. Samples were denaturized (70° C. for 10 min.) before analysis. HiMark HMW (Invitrogen) was used as standard. Electrophoresis was run in XCell Surelock Complete with power station (Invitrogen) for 80 min at 150 V, 120 mA. Gels were stained using SimplyBlue SafeStain from I nvitrogen.

### Carbazole Assay

Heparosan polymers were quantified by carbazol assay according to the method by Bitter T, Muir H M. Anal Biochem 1962 October; 4:330-4.

### Exemplary FVIIIa Activity Assay: Chromogenic Assay

The FVIII activity (FVIII:C) of the rFVIII compound is evaluated in a chromogenic FVIII assay using Coatest SP reagents (Chromogenix) as follows: rFVIII samples and a FVIII standard (e.g. purified wild-type rFVIII calibrated against the 7th international FVIII standard from NIBSC) are diluted in Coatest assay buffer (50 mM Tris, 150 mM NaCl, 1% BSA, pH 7.3, with preservative). Fifty μl of samples, standards, and buffer negative control are added to 96-well microtiter plates (Nunc) in duplicates. The factor IXa/factor X reagent, the phospholipid reagent and CaCl₂ from the Coatest SP kit are mixed 5:1:3 (vol:vol:vol) and 75 μl of this added to the wells. After 15 min incubation at room temperature, 50 μl of the factor Xa substrate S-2765/thrombin inhibitor 1-2581 mix is added and the reagents incubated for 10 minutes at room temperature before 25 μM citric acid, pH 3, is added. The absorbance at 415 nm is measured on a Spectramax microtiter plate reader (Molecular Devices) with absorbance at 620 nm used as reference wavelength. The value for the negative control is subtracted from all samples and a calibration curve prepared by linear regression of the absorbance values plotted vs. FVIII concentration. Specific activity is calculated by dividing the activity of the samples with the protein concentration determined by HPLC. The concentration of the sample is determined by integrating the area under the peak in the chromatogram corresponding to the light chain and compare with the area of the same peak in a parallel analysis of a wild-type unmodified rFVIII, where the concentration is determined by amino acid analyses.

### Exemplary FVIIIa Activity Assay: One-Stage Clot Assay

FVIII activity (FVIII:C) of the rFVIII compounds is evaluated in a one-stage FVIII clot assay as follows: rFVIII samples and a FVIII standard (e.g. purified wild-type rFVIII calibrated against the 7th international FVIII standard from NIBSC) are diluted in HBS/BSA buffer (20 mM hepes, 150 mM NaCl, pH 7.4 with 1% BSA) to approximately 10 U/ml, followed by 10-fold dilution in FVIII-deficient plasma containing VWF (Dade Behring). Samples are subsequently diluted in HBS/BSA buffer. The APTT clot time is measured using an ACL300R or an ACL5000 instrument (Instrumentation Laboratory) using the single factor program. FVIII-deficient plasma with VWF (Dade Behring) is used as assay plasma and SynthASil, (HemoslL™, Instrumentation Laboratory) as aPTT reagent. In the clot instrument, the diluted sample or standard is mixed with FVIII-deficient plasma and aPTT reagents at 37° C. Calcium chloride is added and time until clot formation is determined by measuring turbidity. The FVIII:C in the sample is calculated based on a standard curve of the clot formation times of the dilutions of the FVIII standard.

### Example 1

### Preparation of HEP-Maleimide and HEP-aldehyde polymers

Maleimide and aldehyde functionalized HEP polymers of defined size are prepared by an enzymatic (PmHS1) polymerization reaction using the two sugar nucleotides UDP-GlcNAc and UDP-GlcUA. A priming trisaccharide (GlcUA-GlcNAc-GlcUA)NH₂ is used for initiating the reaction, and polymerization is run until depletion of sugar nucleotide building blocks. The terminal amine (originating from the primer) is then functionalized with suitable reactive groups, in this case either a maleimide functionality designed for conjugation to free cysteines and thioGSC derivatives, or a benzaldehyde functionality designed for reductive amination chemistry to GSC. Size of HEP polymers can be pre-determined by variation in sugar nucleotide: primer stoichiometry. The technique is described in detail in US 2010/0036001. The trisaccharide primer is synthesised as follows:

### Step 1: Synthesis of (2-Fmoc-amino)ethyl 2,3,4-tri-O-acetyl-/3-D-glucuronic acid methyl ester

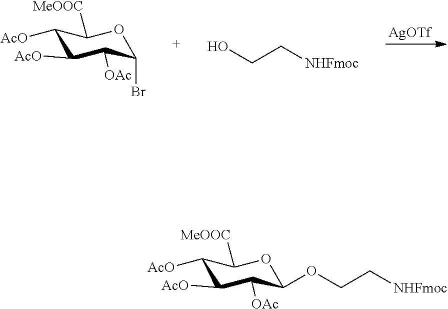

Powdered molecular sieves (1.18 g, 4 Å) were heated at 110° C. in a 50 ml round bottom flask fitted with a magnetic stir bar overnight, flushed with argon, and allowed to cool to room temperature. 900 mg (2.19 mmol) aceto-bromo-β-D-glucuronic acid methyl ester and 748.5 mg (2.64 mmol, 1.2 eq) 2-(Fmoc-amino)ethanol were added under argon, followed by 28 ml dichloromethane. The suspension was stirred for 15 minutes at room temperature and then cooled on an ice/NaCl-slurry for 30 minutes. A white precipitate formed during the cooling process. 676.3 mg (2.63 mmol, 1.2 eq) silver trifluoromethanesulfonate (AgOTf) was added in 3 portions over a period of ˜5 minutes. After 20 minutes the ice-bath was removed. The previously noted white precipitate started dissolving, while at the same time a grey precipitate started to form. The reaction was stirred overnight at room temperature and then quenched by addition of 190 μL triethylamine (2.63 mmol, 1.2 eq). After filtration through a thin Celite 521 pad (˜0.1-0.2 cm deep), and subsequent washing of the filter cake with 20 ml dichloromethane, the combined filtrates were diluted with dichloromethane to 150 ml. The organic phase was washed with 5% NaHCO₃ (1×50 mL) and water (1×50 mL), then dried over magnesium sulfate and filtered. The filtrate was concentrated in vacuo on a rotary evaporator 40° C. water bath) to dryness and then re-dissolved in 2 mL dichloromethane. The solution was injected onto a VersaPak silica gel flash column (23×110 mm, 23 g) and the product eluted with 50% ethyl acetate in hexanes. The product-containing fractions were identified by TLC (ethyl acetate:hexanes, 1:1), and concentrated in vacuo on a rotary evaporator (≦40° C. water bath) to dryness. Trituration of the obtained residue with ˜10 mL diethyl ether yielded the title material as a white crystalline foam. Yield: 293 mg (0.49 mmol, 22.4%).

### Step 2: Synthesis of (2-Fmoc-amino)ethyl β-D-glucuronic acid, sodium salt

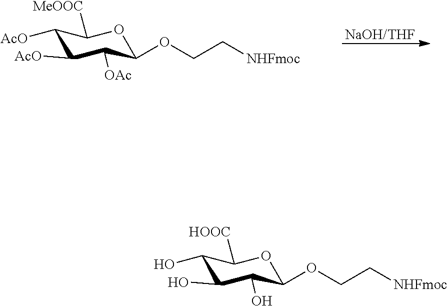

490 mg (0.817 mmol, 1 eq) of (2-Fmoc-amino)ethyl 2,3,4-tri-O-acetyl-/3-D-glucuronic acid methyl ester obtained in step 1 was dissolved in 47.5 mL methanol and 2.5 mL (2.45 mmol, 3 eq) of a 1 M NaOH-solution was slowly added under stirring. The reaction was monitored by TLC using 1-butanol:acetic acid: water=1:1:1 as eluent. After TLC showed complete consumption of the methyl ester, the pH of the reaction mixture was lowered to pH 8-9 by addition of 1 N HCl. 204 mg (2.45 mmol, 3 eq) solid NaHCO₃ followed by 241.7 mg (0.899 mmol, 1.1 eq) Fmoc-chloride was then added. When TLC analysis showed completion of reaction, the reaction mixture was diluted with ˜150 mL water, extracted twice with ethyl acetate (2×30 mL), and then concentrated in vacuo over a 40° C. water bath to about 20 mL to remove any remaining organic solvents. The solution was acidified by addition of acetic acid to a content of ˜5% (v:v), and passed through a 5 gram Strata C-18E SPE tube (pre-wetted in methanol, and equilibrated in 5% acetic acid according to manufacturer's instructions). The resin was washed with 5% acetic acid, and the product was eluted with a mixture of 90% methanol with 10% Tris.HCl, pH 7.2 (v:v). After concentration in vacuo (≦40° C. water bath) to dryness, the residue was redissolved and the pH was adjusted to pH 7.2 with sodium hydroxide. This solution was used directly as stock solution in the synthesis of (2-Fmoc-amino)ethyl 4-O-(2-deoxy-2-acetamido-α-D-glucopyranosyl)β-D-glucuronic acid below without further purification.

### Step 3: Synthesis of (2-Fmoc-amino)ethyl 4-O-(2-deoxy-2-acetamido-a-D-glucopyranosyl) β-D-glucuronic acid, sodium salt

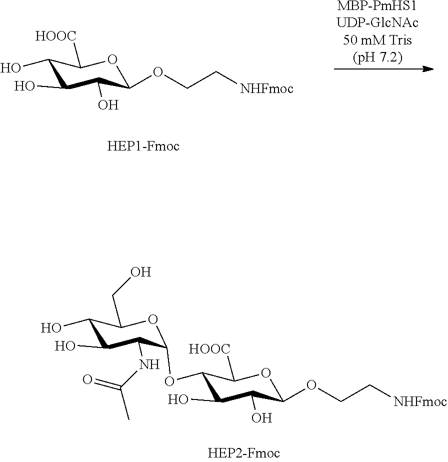

To a solution of 380 mg (2-Fmoc-amino)ethyl/3-D-glucuronic acid obtained in step 2 (0.83 mmole, 1 eq) in 100.8 mL water was added 5.6 mL 1 M Tris-HCl, pH 7.2, 5.6 mL 100 mM MnCl₂, and 1.8 g UDP-GlcNAc (2.79 mmole, 3.4 eq). After slow addition of 5.1 mL MBP-PmHS1 enzyme (15.47 mg/mL; 78.9 mg) over ˜1 min, the reaction was left to stir slowly at room temperature until TLC analysis (1-butanol:acetic acid:water=2:1:1) showed nearly complete conversion of starting material. The solution was acidified by addition of 2.8 mL acetic acid to precipitate the spent MBP-PmHS1 and transferred into 50 mL centrifuge bottles. The solution was then centrifuged for 30 min at 10,000 rpm in a JM-12 rotor (˜16,000×g) at room temperature. The supernatant was decanted and added 160 mL methanol. The pellet was extracted 4×25 mL with a solution of water:methanol:acetic acid=45:50:5 (v:v:v). The combined supernatant and extracts were passed through 2 g Strata-SAX tubes (equilibrated in water:methanol:acetic acid=45:50:5 (v:v:v)) to remove any UDP & UDP-GlcNAc (complete removal required 28 grams of resin). The target molecule was unretained and passed through the resin under these conditions; while the more highly charged UDP & UDP-GlcNAc were retained. The combined eluates were concentrated in vacuo (water batch; ≦40° C.), re-dissolved in water, and the pH was adjusted to pH 7.2 using sodium hydroxide. This solution was used directly in the next step without further purification.

### Step 4: Synthesis of (2-Fmoc-amino)ethyl 4-O-(2-deoxy-2-acetamido-4-O-(β-D-glucopyranosyluronic acid)-α-D-glucopyranosyl)-β-D-glucuronic acid, disodium salt

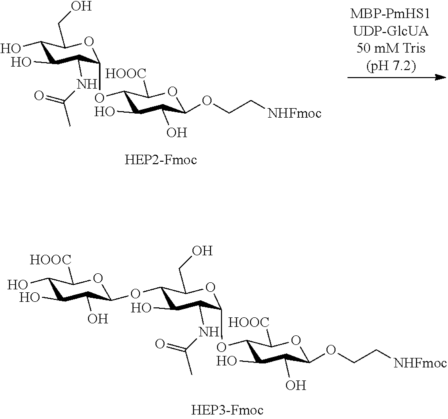

An aqueous solution (38 ml) containing 9 mM (2-Fmoc-amino)ethyl 4-O-(2-deoxy-2-acetamido-α-D-glucopyranosyl)-β-D-glucuronic acid, 30 mM UDP-GlcUA, 50 mM Tris.HCl, and 5 mM MnCl₂ was placed in a spinner flask. Over a period ˜1 min, 9.5 mL MBP-PmHS1 was added dropwise under slow agitation. The reaction mixture was left to stir overnight, after which TLC analysis (eluent: n-BuOH:AcOH:H20=4:1:1 (v:v:v)) showed complete conversion of the starting material. The reaction mixture was filtered through a 1 μm glass fiber syringe filter, and passed through a 5 gram C18-E SPE tube (equilibrated in water, following manufacturer's instructions). The resin was washed with water, followed by elution of the target molecule with a mixture of 90% aqueous MeOH, 1 mM Tris.HCl, pH 7.2. The eluate was concentrated in vacuo (waterbath ≦40° C.), then re-dissolved in 25 mL 10 mM Tris.HCl, pH 7.2, and filtered through a 0.2 μm SFCA syringe filter. The filtrate containing the target molecule was further purified by anion exchange chromatography. An Akta Explorer 100 furnished with a 2.6×13 cm Q Sepharose HP column and operated with Unicorn 5.11 software was used. Two buffer systems (buffer A: 10 mM Tris.HCl, pH 7.2 and buffer B: 10 mM Tris.HCl, pH 7.2, 1 M NaCl) were used for elution. The target molecule was eluted using a 0-20% B gradient over 175 min; at a flowrate of 10 ml/min. 10 ml fraction were collected. The fractions containing product were combined, concentrated on a rotary evaporator in vacuo (waterbath <40° C.) to dryness, and used in the next step without further purification.

### Step 5: Synthesis (2-aminoethyl) 4-O-(2-deoxy-2-acetamido-4-O-β-D-glucopyranosyluronic acid)-α-D-glucopyranosyl)-β-D-glucuronic acid, disodium salt

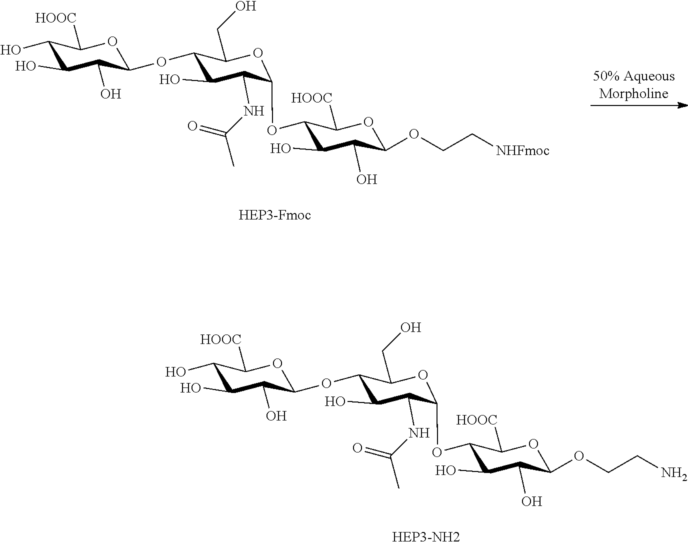

(2-Fmoc-amino)ethyl 4-O-(2-deoxy-2-acetamido-4-O-(β-D-glucopyranosyluronic acid)-α-D-glucopyranosyl)-β-D-glucuronic acid, disodium salt obtained as described in step 4, was dissolved in 4 mL water and cooled on an ice-bath. A volume of 4 mL neat morpholine was added under stirring and the ice bath was removed. Stirring was continued at room temperature, until TLC analysis (n-BuOH:AcOH:H₂O=3:1:1 (v:v:v)) using UV 254 nm detection showed complete consumption of starting material. Reaction was complete within less than 1.5 hrs. The reaction mixture was diluted with ˜50 mL water and extracted three times with 50 mL EtOAc. The aqueous phase containing the target molecule was concentrated on a rotary evaporator in vacuo (waterbath <40° C.) and co-evaporated three times with water. The residue was re-dissolved in 10 mL water and passed through a 1 gram SDB-L SPE column preequilibrated in water. The target passed through the column unretained. The column was washed with 10 mL water and the combined fractions with target were concentrated in vacuo to dryness (water bath; ≦40° C.). The obtained residue was dissolved in 1.5 mL 1 M NaOAc, pH 7.5, filtered through a 0.2 μm spinfilter, and desalted by size-exclusion chromatography over a Sephadex G-10 column (2×75 cm, 235 mL) with water as eluent. Structure of the title material was confirmed by MALDI-TOF MS (matrix: 5 mg/mL ATT; 50% acetonitrile/0.05% trifluoroacetic acid): 636.83 [M+Na⁺]. After lyophilization, the title material was dissolved in water, the pH of the obtained solution was adjusted to pH 7.0-7.5 by addition of sodium hydroxide, and the trisaccharide content was determined by carbazole assay (Bitter T, Muir H M. Anal Biochem 1962 October; 4:330-4). The obtained stock solution was aliquoted and stored at −80° C. in tightly sealed containers until needed.

The overall isolated yield of (2-aminoethyl) 4-O-(2-deoxy-2-acetamido-4-O-(β-D-glucopyranosyluronic acid)-α-D-glucopyranosyl)-β-D-glucuronic acid starting from (2-Fmoc-amino)ethyl β-D-glucuronic acid was 210 mg (0.34 mmole, 41%).

The heparosan polymer is synthesised from the trisaccharide primer as follows:

Production of Heparosan Polysaccharide with amine terminal

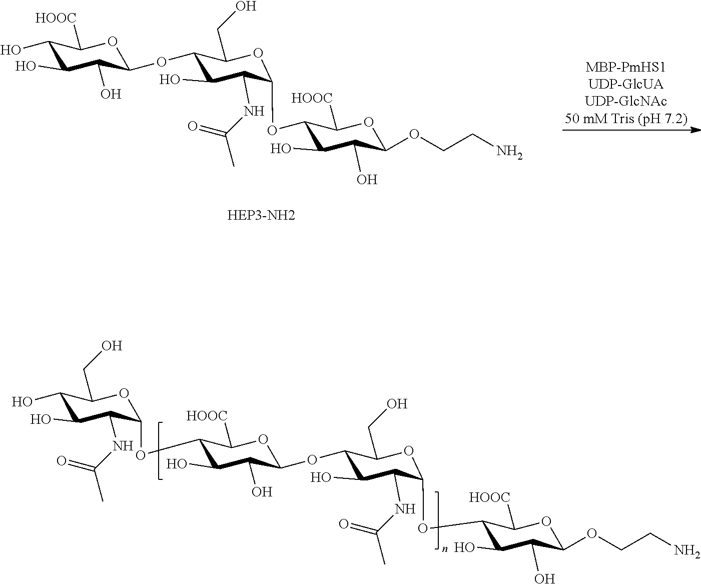

To obtain a heparosan polymer derivative with a free amine group (HEP-NH₂), the Pasteurella multocida heparosan synthase 1 (PmHS1; DeAngelis & White, 2002 J Biol Chem) was used to chemoenzymatically synthesize polymer chains in a parallel fashion in vitro (Sismey-Ragatz et al., 2007 J Biol Chem and U.S. Pat. No. 8,088,604). A fusion of the E. coli maltose-binding protein with PmHS1 was used as the catalyst for elongating the (2-aminoethyl) 4-O-(2-deoxy-2-acetamido-4-O-β-D-glucopyranosyluronic acid)-α-D-glucopyranosyl)-β-D-glucuronic acid (HEP3-NH₂) obtained in step 5 into longer polymer chains using UDP-GlcNAc and UDP-GlcUA precursors and MnCl₂ catalysis as described in US2010036001.

### Synthesis of HEP-Maleimide and HEP-Benzaldehyde Polymers:

HEP-benzaldehydes can be prepared by reacting amine functionalized HEP polymers with a surplus of N-succinimidyl-4-formylbenzoic acid (Nano Letters (2007) 7(8), pp. 2207-2210) in aqueous neutral solution. The benzaldehyde functionalized polymers may be isolated by ion-exchange chromatography, size exclusion chromatography, or HPLC.

HEP-maleimides can be prepared by reacting amine functionalized HEP polymers with a surplus of N-maleimidobutyryl-oxysuccinimide ester (GMBS; Fujiwara, K., et al. (1988) J Immunol Meth 112, 77-83).

More specifically, to obtain a heparosan polymer derivative for coupling via reductive amination, etc. to accessible amino functionalities on the target drug compound, heparosan-NH₂, was coupled with N-succinimidyl-4-formylbenzoic acid, to form a benzaldehyde-modified heparosan polymer. Basically, in one example, N-succinimidyl-4-formylbenzoic acid (Chem-Impex, Inc) dissolved in dimethyl sulfoxide (11.94 mg in 205 mL) was slowly added to a stirred solution of 62.7 g of 43.8 kDa heparosan polymer-NH₂ dissolved in 380 mL 1M sodium phosphate, pH 7.0, 2180 ml water, and 1040 mL dimethylsulfoxide. The reaction mixture was left to stir at room temperature overnight, followed by alcohol precipitation at ambient temperature. The pellet with product was dissolved in 3 L of 500 mM sodium acetate, pH 6.8, further purified and then concentrated by cross flow filtration. The benzaldehyde or maleimide functionalized polymers may alternatively be isolated by ion-exchange chromatography, size exclusion chromatography, or HPLC.

Any HEP polymer functionalized with a terminal primary amine (HEP-NH₂) may be used in the present examples. Two options are shown below:

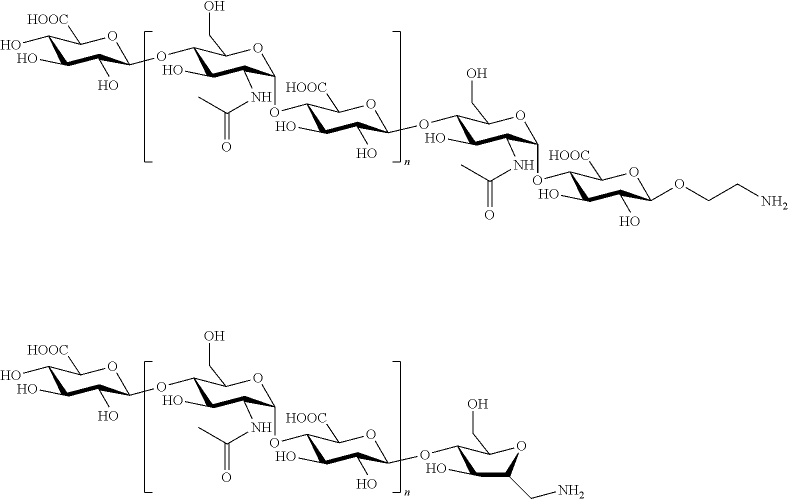

Furthermore the terminal sugar residue in the non-reducing end of the polysaccharide can be either N-acetylglucosamine or glucuronic acid (glucuronic acid is drawn above). Typically a mixture of both is to be expected if equimolar amount of UDP-GlcNAc and UDP-GlcUA has been used in the polymerization reaction. n can be 5-450, such as 50 to 400; 100 to 200; or 150 to 190.

### Example 2

### Synthesis of [(4-mercaptobutanoyl)glycyl]sialic acid cytidine monophosphate (GSC-SH)

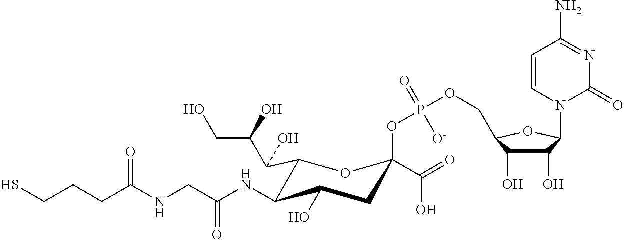

Glycyl sialic acid cytidine monophosphate (200 mg; 0.318 mmol) was dissolved in water (2 ml), and thiobutyrolactone (325 mg; 3.18 mmol) was added. The two phase solution was gently mixed for 21 h at room temperature. The reaction mixture was then diluted with water (10 ml) and applied to a reverse phase HPLC column (C18, 50 mm×200 mm). Column was eluted at a flow rate of 50 ml/min with a gradient system of water (A), acetonitrile (B) and 250 mM ammonium hydrogen carbonate (C) as follows: 0 min (A: 90%, B: 0%, C:10%); 12 min (A: 90%, B: 0%, C:10%); 48 min (A: 70%, B: 20%, C:10%). Fractions (20 ml size) were collected and analysed by LC-MS. Pure fractions were pooled, and passed slowly through a short pad of Dowex 50 W×2 (100-200 mesh) resin in sodium form, before lyophilized into dry powder. Content of title material in freeze dried powder was then determined by HPLC using absorbance at 260 nm, and glycyl sialic acid cytidine monophosphate as reference material. For the HPLC analysis, a Waters X-Bridge phenyl column (5 μm 4.6 mm×250 mm) and a water acetonitrile system (linear gradient from 0-85% acetonitrile over 30 min containing 0.1% phosphoric acid) was used. Yield: 61.6 mg (26%). LCMS: 732.18 (MH⁺); 427.14 (MH⁺-CMP). Compound was stable for extended periods (>12 months) when stored at −80° C.

### Example 3

### Preparation of 38.8 kDa HEP-GSC Reagent (Succinimide Sublinker)

The HEP reagent was prepared by coupling GSC-SH ([(4-mercaptobutanoyl)-glycyl]sialic acid cytidine monophosphate) with HEP-maleimide in a 1:1 molar ratio as follows: to GSC-SH (0.50 mg) dissolved in 50 mM Hepes, 100 mM NaCl, pH 7.0 (50 μl) was added 26.38 mg of the 38.8 kDa HEP-maleimide dissolved in 50 mM Hepes, 100 mM NaCl, pH 7.0 (1350 μl). The clear solution was left for 2 hours at 25° C. The excess of GSC-SH was removed by dialysis, using a Slide-A-Lyzer cassette (Thermo Scientific) with a cut-off of 10 kDa. The dialysis buffer was 50 mM Hepes, 100 mM NaCl, 10 mM CaCl₂, pH 7.0. The reaction mixture was dialyzed twice for 2.5 hours. The recovered material was used as such, assuming a quantitative reaction between GSC-SH and HEP-maleimide. The HEP-GSC reagent made by this procedure will contain a HEP polymer attached to sialic acid cytidine monophosphate via a succinimide linkage.

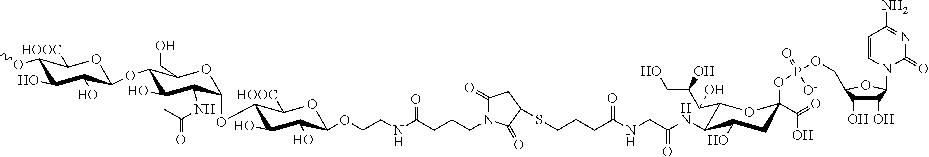

### Example 4

### Preparation of 20 kDa HEP-GSC Reagent (Succinimide Sublinker)

This compound can be prepared using 20 kDa HEP-maleimide and [(4-mercaptobutanoyl)glycyl]sialic acid cytidine monophosphate in a similar way as described for 38.8 kDa HEP-GSC above.

### Example 5

### Preparation of 73 Kda Hep-Gsc Reagent (Succinimide Sublinker)

This compound was prepared using 73 kDa-HEP-maleimide and [(4-mercaptobutanoyl)glycyl]sialic acid cytidine monophosphate in a similar way as described for 38.8 kDa HEP-GSC above.

### Example 6

### General Description for Making 21 kDa, 40 kDa and 73 kDa HEP-GSC Reagents (4-Methylbenzoyl Sublinker)

HEP-benzaldehydes were (optionally) obtained as freeze dried Hepes stabilized powders. GSC prepared according to WO07056191 was dissolved in neutral buffer and added directly to the freeze dried HEP-benzaldehyde. 5-25 equivalents (eq) of GSC were used compared to HEP-benzaldehyde. The liquid solution was gently mixed until all HEP-benzaldehyde was in solution. Then a reducing agent (NaBH₃CN or alternatively boran complex) was added in portions over 2 h time course until a 50 mM solution was obtained. The solution was then transferred to a dialysis chamber (10.000 MWCO) and dialysed against a 500-1000 fold volume of 25 mM Hepes, pH 7.2 twice, for 2 h and 16 h respectively. The inner-chamber was then analysed for GSC remains using Waters X-bridge phenyl (5 μm) 4.6 mm×250 mm (0.1% phosphoric acid—water—acetonitrile system). Upon GSC removal the content of the chamber was freeze dried into a powder containing Hepes-stabilized HEP-GSC.

### Example 7

### Preparation of 41.5 kDa HEP-GSC reagent (4-methylbenzoyl sublinker)

Glycyl sialic acid cytidine monophosphate (GSC) (20 mg; 32 μmol) in 5.0 ml 50 mM Hepes, 100 mM NaCl, 10 mM CaCl₂ buffer, pH 7.0 was added directly to dry 41.5 kDa HEP-benzaldehyde (99.7 mg; 2.5 μmol, carbazole quantification assay). The mixture was gently rotated until all HEP-benzaldehyde had dissolved. During the following 2 hours, a 1M solution of sodium cyanoborohydride in MilliQ water was added in portions (5×50 μl), to reach a final concentration of 48 mM. Excess of GSC was then removed by dialysis as follows: the total reaction volume (5250 μl) was transferred to a dialysis cassette (Slide-A-Lyzer Dialysis Cassette, Thermo Scientific Prod#66810 with cut off 10 kDa capacity: 3-12 ml). Solution was dialysed for 2 hours against 2000 ml of 25 mM Hepes buffer (pH 7.2) and once more for 17 h against 2000 ml of 25 mM Hepes buffer (pH 7.2). Complete removal of excess GSC from inner chamber was verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using GSC as reference. Inner chamber material was collected and freeze dried to give 83% (carbazole quantification assay) 41.5 kDa HEP-GSC as white powder.

The HEP-GSC reagent prepared according to this procedure contained a HEP polymer attached to GSC via a methylbenzoyl linkage.

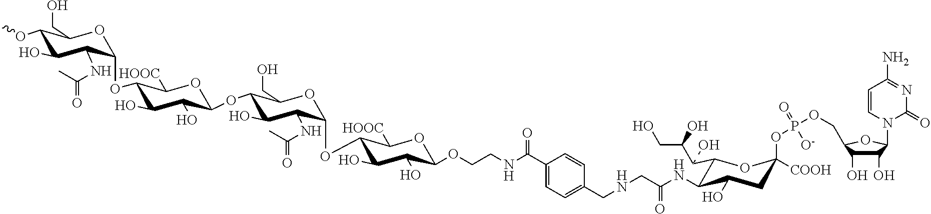

### Example 8

### Preparation of 21 kDa HEP-GSC reagent

This compound was prepared using 21 kDa HEP-aldehyde and glycyl sialic acid cytidine monophosphate (GSC) in a similar way as described for 41.5 kDa HEP-GSC above. Yield was 78% after freeze drying.

### Example 9

### Preparation of 73 kDa HEP-GSC reagent

This compound was prepared using 73 kDa HEP-aldehyde glycyl sialic acid cytidine monophosphate (GSC) in a similar way as described for 41.5 kDa HEP-GSC above. Yield was 70% after freeze drying.

### Example 10

### Reduction of FVIII-K1804C

FVIII-K1804C when produced in mammalian cells, is isolated with its C1804 cysteine blocked as mixed disulfides by low molecular thiols. To facilitate HEP conjugation, the protein has initially to be deblocked in order to make the C1804 thiol group available for coupling. Deblocking is performed by chemical reduction using the phosphine-based reducing as follows: FVIII-K1804C (15.6 mg) was incubated with Tris(3-sulfophenyl)phosphine(42 mg) for 4.5 h at 5° C. in 15.5 ml of 20 mM Imidazol, 10 mM CaCl2, 1 M glycerol, 0,02% Tween80, 1 M NaCl, pH 7.3 (imidazole buffer). Reaction mixture was divided in three portions and each diluted with 15 ml of imidazole buffer, before transferring to an ultrafiltration tube (Millipore Amicon Ultra, cut off 10 kD). Sample volume was reduced by centrifugation, but not to less than 5 ml to avoid protein precipitation. Fresh buffer was added, and centrifugation dilution step was repeated two more times. The combined samples were diluted to 45 ml with loading buffer (20 mM Imidazol, 10 mM CaCl2, 0,02% Tween80, 25 mM NaCl, 1 M glycerol, pH 7.3) and applied to a 1 ml MonoQ 5/50 GL ion-exchange column (Amersham Biosciences, GE Healthcare) equilibrated in loading buffer. After wash with 2 column volume of loading buffer A to remove unbound protein, FVIII K1804C was eluted in one step with buffer B (20 mM Imidazol, 10 mM CaCl2, 0,02% Tween80, 1 M NaCl, 1 M glycerol, pH 7.3). Fractions containing FVIII K1804C were pooled, and applied to a HiLoad 16/60 Superdex 200 prep grad column (CV 124 ml) equilibrated in elution buffer (20 mM Imidazol, 10 mM CaCl2, 0.02% Tween 80, 1 M NaCl, 1 M glycerol, pH 7.3). FVIII K1804C was then eluted in same elution buffer. Fractions were concentrated using by ultrafiltration (Millipore Amicon Ultra, cut off 10 kD). 9.0 mg de-protected FVIII K1804C was isolated in 7 ml 20 mM Imidazol, 10 mM CaCl2, 0,02% Tween80, 1 M NaCl, 1 M glycerol, pH 7.3 (1,29 mg/ml) as determined by RP-HPLC.

### Example 11

### Preparation of 52k-HEP-[C]-FVIII K1804C

FVIII-K1804C (6.3 mg) reduced as described above was reacted with 52k-HEP-maleimide (5.7 mg) in 4.9 ml of 20 mM Imidazol, 10 mM CaCl2, 1 M glycerol, 0,02% Tween80, 1 M NaCl, pH 7.3 for 20 hours at room temperature. Reaction mixture was diluted to 45 ml with loading buffer (50 mM Hepes, 10 mM CaCl2, 100 mM NaCl, pH 7.0) and applied to a 1 ml MonoS 5/50 GL ion-exchange column (Amersham Biosciences, GE Healthcare) equilibrated in loading buffer. Unbound protein was washed out using 10 column volumes of 50 mM Hepes, 10 mM CaCl2, 100 mM NaCl, pH 7.0. 52k-HEP-[C]-FVIII K1804C was eluted with 20 column volumes of a 80% A (50 mM Hepes, 10 mM CaCl2, 100 mM NaCl, pH 7.0) and 20% B (50 mM Hepes, 10 mM CaCl2, 1 M NaCl, pH 7.0) buffer mixture. A mixture of 52k-HEP-[C]-FVIII K1804C and unconjugated FVIII K1804C could be obtained by subsequent step elution with 10 column volumes of a 50% A (50 mM Hepes, 10 mM CaCl2, 100 mM NaCl, pH 7.0) and 50% B (50 mM Hepes, 10 mM CaCl2, 1 M NaCl, pH 7.0) buffer mixture. Pure fractions were identified by HPLC, before being pooled and applied to a HiLoad 16/60 Superdex 200 prep grad column (CV 124 ml) equilibrated in 10 mM Histidine, 2 mM CaCl2, 25 mM NaCl, 0.01% Tween 80, 8.8 mM sucrose pH 7.0. Column was eluted in same buffer, and fractions containing product were pooled and concentrated using by ultrafiltration (Millipore Amicon Ultra, cut off 10 kD) to give 2.2 mg of 52k-HEP-[C]-FVIII K1804C in 7 ml 10 mM Histidine, 2 mM CaCl2, 25 mM NaCl, 0.01% Tween 80, 8.8 mM sucrose pH 7.0.

### Example 12

### Preparation of 27k-HEP-[C]-FVIII K1804C

This conjugate was prepared as described above, using FVIII-K1804C (4.30 mg) and 27k-HEP-maleimide (5.41 mg). 2.46 mg (56%) 27k-HEP-[C]-FVIII K1804C was isolated in 7 ml of 10 mM Histidine, 2 mM CaCl2, 25 mM NaCl, 0.01% Tween 80, 8.8 mM sucrose pH 7.0

### Example 13

### Preparation of 73k-HEP-[C] FVIII K1804C

This conjugate was prepared as described above, using FVIII-K1804C (4.0 mg) and 73k-HEP-maleimide (5.8 mg). 1.48 mg (37%) 73k-HEP-[C]-FVIII K1804C was isolated in 7 ml of 10 mM Histidine, 2 mM CaCl2, 25 mM NaCl, 0.01% Tween 80, 8.8 mM sucrose pH 7.0

### Example 14

### Preparation of 108k-HEP-[C] FVIII K1804C

FVIII-K1804C (5.8 mg) reduced as described above was reacted with 108k-HEP-maleimide (27.0 mg) in 5.7 ml of 20 mM Imidazol, 10 mM CaCl2, 1 M glycerol, 0,02% Tween80, 1 M NaCl, pH 7.3 for 16 hours at room temperature. Reaction mixture was diluted to 50 ml with 20 mM imidazol, 10 mM CaCl2, 0,02% Tween80, 25 mM NaCl, 1 M glycerol, pH 7.3 and applied to a 1 ml MonoQ 5/50 GL ion-exchange column (Amersham Biosciences, GE Healthcare) equilibrated in buffer A (20 mM imidazol, 10 mM CaCl2, 0,02% Tween80, 25 mM NaCl, 1 M glycerol, pH 7.3). Unbound protein was washed out using 10 column volumes of buffer A. Column was then eluted with a 0-35% gradient 10 column volumes buffer B (20 mM Imidazol, 10 mM CaCl2, 0,02% Tween80, 25 mM NaCl, 1 M glycerol, pH 7.3) followed by an additional 10 column volumes of 35% B buffer. Pure fractions were identified by HPLC, pooled and applied to a HiLoad 16/60 Superdex 200 prep grad column (CV 124 ml) equilibrated in 10 mM Histidine, 2 mM CaCl2, 25 mM NaCl, 0.01% Tween 80, 8.8 mM sucrose pH 7.0. Column was eluted in same buffer, and fractions containing product were collected and concentrated by ultrafiltration (Millipore Amicon Ultra, cut off 10 kD) to give 1.40 mg (24%) of 108k-HEP-[C]-FVIII K1804C in 7 ml 10 mM Histidine, 2 mM CaCl2, 25 mM NaCl, 0.01% Tween 80, 8.8 mM sucrose pH 7.0.

### Example 15

### Preparation of 157 kDa HEP-[C]-FVIII K1804C

This conjugate was prepared as described for 108 kDa HEP-[C]-FVIII K1804C, using FVIII-K1804C (2.86 mg) and 157k-HEP-maleimide (20 mg). 0.55 mg (19%) 157k-HEP-[C]-FVIII K1804C was isolated in 7 ml of 10 mM Histidine, 2 mM CaCl2, 25 mM NaCl, 0.01% Tween 80, 8.8 mM sucrose pH 7.0

### Example 16

### Preparation of asialo FVIII

FVIII (28.2 mg) in 6 ml 20 ml imidazol, 10 mM CaCl2, 1M glycerol, 0.02% Tween80, 600 mM NaCl, 7.3 was added sialidase (Arthrobacter ureafaciens, 50 ug, 242 U/mg) and incubated for 1 h at 25° C. One third of the reaction mixture was then loaded on a 1 ml MonoQ 5/50 GL ion-exchange column (Amersham Biosciences, GE Healthcare) equilibrated in buffer A (20 mM imidazol, 10 mM CaCl2, 0,02% Tween80, 25 mM NaCl, 1 M glycerol, pH 7.3). Unbound protein was washed out using 2 column volumes of buffer A. Column was then eluted with a 0-20% gradient 5 column volumes of buffer B (20 mM Imidazol, 10 mM CaCl2, 0,02% Tween80, 1 M NaCl, 1 M glycerol, pH 7.3) followed by 10 column volumes of 20% B buffer to elute sialidase. Asialo FVIII was then eluted with 10 column volumes of 100% buffer B. The chromatographic separation was repeated two times more—each time with one third of the reaction mixture. Fractions containing pure protein were combined to give 24.5 mg asialo FVIII in 6 ml 20 ml imidazol, 10 mM CaCl2, 1M glycerol, 0.02% Tween80, 1 M NaCl, 7.3

### Example 17

### Preparation of 38.8 kDa HEP-[O]-FVIII

Asialo FVIII (10 mg) in 2.45 ml 20 mM imidazol, 10 mM CaCl2, 1M glycerol, 0.02% Tween80, 1 M NaCl, 7.3 was added 38.8k-HEP-GSC (8.46 mg) obtained from example 3 in 1 ml 50 mM HEPES, 100 mM NaCl, 10 mM CaCl2, pH 7.0 and ST3GaII (1.44 mg, 21.6 U/mg in 600 ul 50 mM Tris, 100 mM NaCl pH 8.0). The reaction mixture was incubated at 32° C. for 17 h. N-Acetylneuraminic acid cytidine monophosphate (134 ul of a 156 mM solution in 20 mM imidazol, 10 mM CaCl2, 1M glycerol, 0.02% Tween80, 1 M NaCl, 7.3) was added together with ST3GaIIII (1 mg, 1.1 U/mg in 1.40 ml of 20 mM Hepes, 120 mM NaCl, 50% glycerol, pH 7.0) and incubation was continued for an additional hour. The entire reaction mixture was then loaded onto a 1 ml MonoS 5/50 GL ion-exchange column (Amersham Biosciences, GE Healthcare) equilibrated in buffer A (50 mM hepes, 10 mM CaCl2, 0.02% Tween80, 100 mM NaCl, pH 7.0). Unbound protein was eluted with 12 column volumes of buffer A, and HEP modified FVIII was eluted with 20% buffer B (50 mM hepes, 10 mM CaCl2, 0.02% Tween80, 1M NaCl, pH 7.0). The fractions containing HEP modified FVIII were identified by HPLC, pooled and applied to a HiLoad 16/60 Superdex 200 prep grad column (CV 124 ml) equilibrated in 10 mM Histidine, 2 mM CaCl2, 25 mM NaCl, 0.01% Tween 80, 8.8 mM sucrose pH 7.0. Column was eluted in same buffer and fractions containing product were collected to give 2.48 mg (25%) of 38.8k-HEP-[O]-FVIII in 12 ml 10 mM Histidine, 2 mM CaCl2, 25 mM NaCl, 0.01% Tween 80, 8.8 mM sucrose pH 7.0, as quantified by HPLC.

### Example 18

### Preparation of 73 kDa HEP-[O]-FVIII

This compound was prepared in almost similar way as for the 38.8 kDa HEP-[O]-FVIII. Asialo FVIII (10 mg) in 2.45 ml 20 mM imidazol, 10 mM CaCl2, 1M glycerol, 0.02% Tween80, 1 M NaCl, 7.3 was added 73 kDa-HEP-GSC (15.35 mg) obtained from example 5 in 1 ml 50 mM HEPES, 100 mM NaCl, 10 mM CaCl2, pH 7.0 and ST3GaII (1.44 mg, 21.6 U/mg in 600 ul 50 mM Tris, 100 mM NaCl pH 8.0). The reaction mixture was incubated at 32° C. for 20 h. N-Acetylneuraminic acid cytidine monophosphate (134 ul of a 156 mM solution in 20 mM imidazol, 10 mM CaCl2, 1M glycerol, 0.02% Tween80, 1 M NaCl, 7.3) was added together with ST3GaIIII (1 mg, 1.1 U/mg in 1.40 ml of 20 mM Hepes, 120 mM NaCl, 50% glycerol, pH 7.0) and incubation was continued for an additional 30 min. The reaction mixture was loaded onto a 1 ml MonoS 5/50 GL ion-exchange column (Amersham Biosciences, GE Healthcare) equilibrated in buffer A (50 mM hepes, 10 mM CaCl2, 0.02% Tween80, 100 mM NaCl, pH 7.0). Unbound protein was eluted with 12 CV of buffer A. Column was then step eluted with 10 CV of 20% buffer B (50 mM hepes, 10 mM CaCl2, 0.02% Tween80, 1M NaCl, pH 7.0) giving pure 73 kDa HEP-[O]-FVIII. Fractions were combined, and applied to a HiLoad 16/60 Superdex 200 prep grad column (CV 124 ml) equilibrated in 10 mM Histidine, 2 mM CaCl2, 25 mM NaCl, 0.01% Tween 80, 8.8 mM sucrose pH 7.0. Column was eluted in same buffer and fractions containing product were collected to give 1.23 mg (12%) of 73k-HEP-[O]-FVIII in 6.7 ml 10 mM Histidine, 2 mM CaCl2, 25 mM NaCl, 0.01% Tween 80, 8.8 mM sucrose pH 7.0, as quantified by HPLC.

### Example 19

### Preparation of 73 kDa-HEP-[N]-FVIII

This material was only prepared on an analytical scale. Asialo FVIII (20 ug) in 5 ul 20 mM imidazol, 10 mM CaCl2, 1M glycerol, 0.02% Tween80, 1 M NaCl, 7.3 was in 4 different experiments added a solution of 73 kDa-HEP-GSC (2 eq. (17 ug, 9.4 ul); 4 eq. (34 ug; 19 ul); 8 eq. (66 ug; 38 ul); and 20 eq. (165 ug; 94 ul)) in 50 mM HEPES, 100 mM NaCl, 10 mM CaCl2, pH 7.0 respectively. To all samples were then added 4 ug ST3GaIII (1.1 U/mg in 5.7 ul of 20 mM Hepes, 120 mM NaCl, 50% glycerol, pH 7.0). For all 4 reactions, the final volume was then adjusted to 18.3 ul using 50 mM Hepes, 100 mM NaCl, 10 mM CaCl2, pH 7.0. Reaction mixtures were incubated 28 hours at 32° C., after which, mono- and poly conjugated 73 kDa-HEP-[N]-FVIII clearly was observed by subsequent SDS-PAGE analysis (FIG. 4).

### Example 20

### Preparation of 41.5 kDa-HEP-[O]-FVIII

FVIII was concentrated to 5,9 mg/mL and buffer-exchanged with 20 mmol/kg Histidine+500 mmol/kg NaCl+10 mmol/kg CaCl2+2.1 mol/kg Glycerol, pH6.1, in Amicon Ultra centrifugal filters, Ultracel-30K, (Millipore). The GSC-HEP was dissolved in the same buffer and buffer exchanged using dialysis with Slide-A-Lyzer dialysis cassettes, 10.000MWCO (Thermo Scientific), giving 15.6 mg/mL in final concentration.

18.2 mg of FVIII was mixed with 16 ug of Sialidase, Athrobactor ureafaciens, 0,6 mg of ST3GaII, porcine, and 7,6 mg of 41.5 kDa GSC-HEP. The components were mixed gently and incubated at room temperature for 16 hours.

The solution was diluted 1:9 with 20 mmol/kg Histidine+10 mmol/kg CaCl2+2 mol/kg Glycerol+0,05% (w/w) Poloxamer 188, pH6,1 and loaded to a column packed with Source 30Q (GE Healthcare Bio-Sciences), 20 mL resin with 10 cm bedheight. The column was previously equilibrated with 20 mmol/kg Histidine+10 mmol/kg CaCl2+50 mmol/kg NaCl+2 mol/kg Glycerol+0,05% (w/w) Poloxamer 188, pH6,1, and the HEP-N8 was eluted with a gradient over 50CV from equilibration buffer to 20 mmol/kg Histidine+10 mmol/kg CaCl2+500 mmol/kg NaCl+2 mol/kg Glycerol+0,05% (w/w) Poloxamer 188, pH6.1. The fractions with 41.5 kDaHEP-[O]-N8 were pooled and concentrated to 1,2 mg/mL using Amicon Ultra centrifugal filters, Ultracel-30K (Millipore).

The 41.5 kDa HEP-[O]-N8, 4.8 mg, was mixed with 81 ug ST3GaIIII, rat, and 2.6 mg CMP-NAN. The solution was gently mixed and incubated at room temperature for 16 hours.

The solution was applied to a column packed with TSK Phenyl-5PW, 20 um (Tosoh Bioscience), 1 mL resin with 5 cm bedheight, which was equilibrated with 20 mmol/kg Histidine+10 mmol/kg CaCl2+450 mmol/kg NaCl+1 mol/kg Glycerol+0.05% (w/w) Poloxamer 188, pH6.1 prior to application. The 41.5 kDa-HEP-[O]-N8 does not bind to the resin and was collected in the flow through. The ST3GaI3 binds to the resin and was separated from 41.5 kDa-HEP-[O]-N8.

The solution with 41.5 kDaHEP-[O]-N8 was applied to a column packed with Superdex 200 pg (GE Healthcare Bio-Sciences) 120 mL resin with 60 cm bedheight. The column was equilibrated with 37.5 mmol/kg Histidine+1,5 mmol/kg Methionine+6.6 mmol/kg CaCl2+600 mmol/kg NaCl+34 mmol/kg sucrose+0,05% (w/w) Poloxamer 188, pH6,1, which was also used as buffer during the run. The fractions contain 41.5 kDaHEP-[O]-N8 were pooled and concentrated to 0.4 mg/mL with Amicon Ultra centrifugal filters, Ultracel-30K, (Millipore).

### Example 21

### Synthesis of Neuraminic Acid Cytidine Monophosphate Based 41.5 kDa HEP conjugates with 4-methylbenzoyl linkage

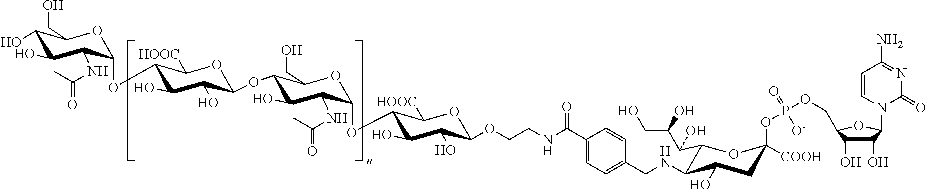

Neuraminic acid cytidine monophosphate is produced as described in Eur. J. Org. Chem. 2000, 1467-1482. Reaction with HEP-aldehyde is performed as described in example 7, replacing GSC with neuraminic acid cytidine monophosphate. Neuraminic acid cytidine monophosphate (32 μmol) is dissolved in 50 mM Hepes, 100 mM NaCl, 10 mM CaCl₂ buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of neuraminic acid cytidine monophosphate is then removed by dialysis as described in example 7. Complete removal of neuraminic acid cytidine monophosphate from inner chamber is verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using neuraminic acid cytidine monophosphate as reference. Inner chamber material is then collected and freeze dried. The reagent made by this procedure contains a HEP polymer attached to sialic acid cytidine monophosphate via a 4-methylbenzoyl linkage.

### Example 22

### Synthesis of 9-Amino-9-Deoxy-N-Acetylneuraminic Acid Cytidine Monophosphate Based HEP Conjugates with 4-Methylbenzoyl Linkage

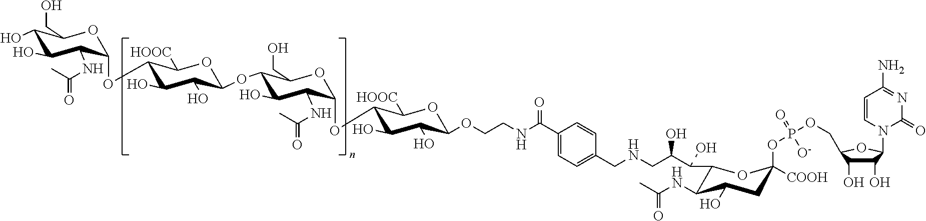

9-deoxy-amino N-acetylneuraminic acid cytidine monophosphate is produced as described in Eur. J. Biochem 168, 594-602 (1987). Reaction with HEP-aldehyde is performed as described in example 7, replacing GSC with 9-amino-9-deoxy-N-acetylneuraminic acid cytidine monophosphate. 9-Amino-9-deoxy-N-acetylneuraminic acid cytidine monophosphate (32 μmol) is dissolved in 50 mM Hepes, 100 mM NaCl, 10 mM CaCl₂ buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of 9-amino-9-deoxy-N-acetylneuraminic acid cytidine monophosphate is then removed by dialysis as described in example 7. Complete removal of 9-amino-9-deoxy-N-acetylneuraminic acid cytidine monophosphate from inner chamber is verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using 9-amino-9-deoxy-N-acetylneuraminic acid cytidine monophosphate as reference. Inner chamber material is collected and freeze dried. The reagent made by this procedure contains a HEP polymer attached to sialic acid cytidine monophosphate via a 4-methylbenzoyl linkage and is suitable for glycoconjugation with an asialo FVIII glycoprotein.

### Example 23

### Synthesis of 2-Keto-3-Deoxy-Nonic Acid Cytidine Monophosphate Based HEP Conjugates with 4-Methyl Benzoyl Linkage

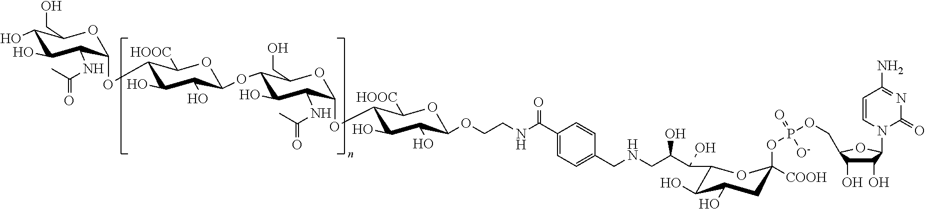

In a way similar to that shown in examples 19 and 20 HEP-sialic acid cytidine monophosphate reagent can be made starting from the sialic acid KDN. The initial amino derivatization at the 9-position is performed as described in Eur. J. Org. Chem. 2000, 1467-1482. Reaction with HEP-aldehyde is performed as described in example 7, replacing GSC with 9-amino-9-deoxy-2-keto-3-deoxy-nonic acid cytidine monophosphate. 9-amino-9-deoxy-2-keto-3-deoxy-nonic acid cytidine monophosphate (32 μmol) is dissolved in 50 mM Hepes, 100 mM NaCl, 10 mM CaCl₂ buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of 9-amino-9-deoxy-2-keto-3-deoxy-nonic acid cytidine monophosphate is then removed by dialysis as described in example 7. Complete removal of 9-amino-9-deoxy-N-acetylneuraminic acid cytidine monophosphate from inner chamber is verified by HPLC on Waters X-Bridge phenyl column (4.6 mm×250 mm, 5 pm) and a water acetonitrile system (linear gradient from 0-85% acetonitrile over 30 min containing 0.1% phosphoric acid) using 9-amino-9-deoxy-2-keto-3-deoxy-nonic acid cytidine monophosphate as reference. Inner chamber material is collected and freeze dried. The reagent made by this procedure contains a HEP polymer attached to sialic acid cytidine monophosphate via a 4-methylbenzoyl linkage and is suitable for glycoconjugation with an asialo-FVIII glycoprotein.

### Example 24

### FVIII Activity of Heparosan-Conjugated FVIII

The FVIII activity (FVIII:C) of heparosan-conjugated FVIII 40K-HEP-[O]-N8 was assessed using a two-stage chromogenic assay (Coamatic® Factor VIII kit, Chromogenix) after pre-diluting to approximately 10 IU/mL in HBS/BSA (20 mM hepes, 150 mM NaCl, pH 7.4, supplemented with 1% bovine serum albumin) followed by 10-fold dilution FVIII-deficient plasma with normal level of VWF (Siemens). The calibrator (WHO 8^{th} IS) was reconstituted and diluted 9.4-fold in the plasma. Samples and calibrators were diluted to 20 mU/ml in diluent from the kit and subsequently to 5-4-3-2-1-0.5 and 0.25 mU/mL. Samples, calibrators and a negative control (diluent) were incubated with the FX/FIXa/(pro)thrombin/phospholipid/calcium reagents for 200s at 37° C., before adding stop reagent and FXa substrate. Absorbance at 405 nm was measured continuously for 5 min on a Spectramax plate reader (Molecular Device). A linear plot of ΔA405/min versus FVIII:C of the calibrator was used by the SoftMax Pro 5.4.1 software to calculate FVIII:C of the samples.

The specific activity was calculated by dividing the activity of the samples with the protein concentration determined by reverse-phase high performance liquid chromatography (RP-HPLC) as described (Thim L et al. Haemophilia 2010; 16: 349-359). The concentration of FVIII was determined by comparing the area of the peaks with those of a known amount of non-conjugated FVIII. Only the protein content—and not heparosan—is included in the concentration determination. The specific activity of 40K-HEP-[O]-N8 was calculated to 11850±850 IU/mL (mean and standard deviation of n=3).

### Example 25

### FVIII:C of 40K-HEP-[O]-N8 Added to Haemophilia A Plasma

Post-administration samples were simulated by adding 40k-HEP-[O]-N8 to severe haemophilia A plasma (George King BioMed Inc) to 0.2; 0.6; and 0.9 IU/mL based on activity determined in chromogenic assay. FVIII:C was measured on an ACL TOP 500 instrument (Instrumentation laboratories) with seven different aPTT reagents (see Table 2, below). Human plasma (Siemens) was used as calibrator. The measured FVIII:C was in general within +/−25% of the nominal values (Table 1). At low concentration (0.2 IU/mL), there was a tendency of overestimating FVIII:C, while the measured FVIII:C is closer to the nominal values for the samples containing 0.6 and 0.9 IU/mL 40K-HEP-[O]-N8. Notably, no major differences in FVIII:C was observed with the different aPTT reagents.

TABLE 2
FVIII: C of 40K-HEP-[O]-N8 in clot assay with different aPTT reagents

| | | Measured FVIII: C (% of nominal) | | |
|---|---|---|---|---|
| | | 0.2 IU/mL | 0.6 IU/mL | 0.9 IU/mL |
| aPTT reagent | Manufacturer | nominal | nominal | nominal |
| APTT-SP | IL | 105 ± 19 | 89 ± 14 | 85 ± 13 |
| SynthASil | IL | 126 ± 23 | 100 ± 14 | 88 ± 11 |
| Actin FS | Siemens | 121 ± 10 | 110 ± 9 | 96 ± 4 |
| CK Prest | Stago | 134 ± 12 | 110 ± 11 | 96 ± 8 |
| Pathromtin SL | Siemens | 157 ± 51 | 101 ± 17 | 94 ± 15 |
| Cephascreen | Stago | 111 ± 8 | 106 ± 10 | 95 ± 10 |
| STA-PTT Automate 5 | Stago | 128 ± 15 | 103 ± 9 | 92 ± 7 |

### Example 26

### FVIII Cofactor Activity, Rate of Activation by Thrombin and FVIIIa Decay and Inactivation Analysed by Enzyme Kinetics

The rate of activation by thrombin and the co-factor activity of activated 40k-HEP-[O]-N8 was characterised by studying factor IXa (FIXa)-catalysed activation of factor X (FX) in a purified system containing phospholipids and calcium as described (Christiansen MLS et al. Haemophilia 2010; 16: 878-887), with the modification that in titration of FX to determine Km and Kcat of FX activation, the final concentrations of activated FVIII and FIXa were 5 nM (nominal) and 0.02 nM, respectively. Additionally, the spontaneous decay of activated 40k-HEP-[O]-N8 as well as inactivation by activated protein C (APC) was determined. Non-conjugated FVIII (N8/turoctocog alfa) was included as comparator. The data shown in Table 3, below demonstrates that the kinetic parameters of FVIII activation by thrombin, FVIIIa co-factor function in FIXa-catalysed FX activation and APC-mediated inactivation as well as spontaneous FVIIIa decay were not statistically different for 40k-HEP-[O]-N8 and turoctocog alfa. This indicates that 40k-HEP-[O]-N8 has maintained full FVIII activity.

TABLE 3
Functional properties of 40k-HEP-[O]-N8 measured by enzyme kinetics.
Data are mean and standard deviation of five independent experiments

Rate of

| | | |
|---|---|---|
| Rate of activation | | APC- |
| by thrombin | FVIIIa | mediated |

| | (pM × min⁻¹) | | Cofactor activity | | | decay | inactivation |
|---|---|---|---|---|---|---|---|
| FVIII | Without | With | K_{1/2}FIXa | Kₘ | k_{cat} | constant | of FVIIIa |
| compound | VWF | VWF | (nM) | (nM) | (s⁻¹) | (min⁻¹) | (min⁻¹) |
| 40k-HEP- | 4.2 ± 0.5 | 14.4 ± 1.7 | 1.8 ± 0.1 | 11.8 ± 1.0 | 8.1 ± 0.2 | 0.16 ± 0.04 | 0.17 ± 0.03 |
| [O]-N8 | | | | | | | |
| Turoctocog | 4.0 ± 0.5 | 14.7 ± 1.3 | 1.9 ± 0.2 | 12.0 ± 1.0 | 8.1 ± 0.2 | 0.15 ± 0.04 | 0.20 ± 0.03 |
| alfa/N8 | | | | | | | |

### Example 27

### Haemostatic Effect in Thrombin Generation Assay

The haemostatic effect of 40k-HEP-[O]-N8 in human haemophilia A plasma was evaluated in a thrombin generation assay employing plasma from haemophilia A patients supplemented with normal human platelets. The platelets were isolated from human platelet-rich plasma (PRP) prepared from citrate-stabilized peripheral blood from normal donors. The blood was acidified by adding one volume of acetate citrate dextrose (ACD, 85 mM tri-sodium citrate, 71 mM citric acid and 111 mM glucose) to five volumes of blood and centrifuged 20 min at 220×g. The PRP was transferred to a new tube before centrifuging 15 min at 500×g. The pellet was gently resuspended in 10 ml Hepes-Tyrodes buffer (15 mM HEPES, 138 mM NaCl, 2.7 mM KCl, 1 mM MgCl₂, 5 mM CaCl₂, 5.5 mM dextrose and 1 mg/mL BSA, pH 6.5) with 5 pg/mL prostaglandin E₁ (Sigma) added. After 15 min centrifugation at 500×g was the pellet gently resuspended in 0.5 mL Hepes-Tyrodes buffer and the platelet density determined on a Medonic cell counter (Boule). The platelets were added to severe haemophilia A plasma (George King Bio-Medical Inc.) to 150×10⁹/L (final density 100×10⁹/L). For each sample, 80 μl of this mimicked haemophilia A PRP was mixed with 10 μl FVIII (final concentration 1; 0.3; and 0.1 IU/mL based on activity in chromogenic assay) in HBS/BSA (20 mM Hepes, 150 mM NaCl, 2% BSA, pH 7.4) and 10 μl PRP reagent (Thrombinoscope) and prewarmed 10 min at 37° C. in a Fluoroskan Ascent plate reader (Thermo Electron Corporation). FluCa reagent containing a fluorescent substrate and calcium (Thrombinoscope, 20 μl) was added and emission at 460 nm after excitation at 390 nm was measured continuously for 120 min. The fluorescence signal was corrected for α₂-macroglobulin-bound thrombin activity and converted to thrombin concentration by use of a calibrator (Thrombinoscope) and Thrombinoscope version 5.0.0 software (Synapse BV). The parameters lag-time, time to peak thrombin, peak thrombin, maximal rate of thrombin generation (“Velindex”) and total thrombin activity, corresponding to area under the curve (ETP, endogenous thrombin potential) were calculated by the software. Maximal rate of thrombin generation was additionally determined by linear regression of the part of the thrombin generation curve with steepest increase in thrombin activity using GraphPad Prism version 6.03 software. Parameters from a representative example are shown in Table 4, below. In the absence of FVIII only a small amount of thrombin was formed (and consequently it was not possible to calculate the ETP). Addition of 40k-HEP-[O]-N8 or FVIII starting material (turoctocog alfa, N8) both improved thrombin generation in a dose-dependent manner, seen as shortening of the lag-time and time to peak thrombin, and increase of peak thrombin level, ETP and maximal rate of thrombin generation (Velindex and slope). The effect of 40k-HEP-[O]-N8 and turoctocog alfa were comparable indicating that 40k-HEP-[O]-N8 is fully active in human haemophilia A plasma

TABLE 4
Thrombin generation in human haemophilia A plasma supplemented with normal
human platelets. Data are representative of three individual experiments.

| | | | Time to | | | | | |
|---|---|---|---|---|---|---|---|---|
| FVIII | Conc | Lag time | peak | Peak | ETP | Velindex | Slope | |
| compound | IU/ml | min | min | nM | nM × min | nM/min | nM/min | +/- error |
| Turoctocog | 1.00 | 9.3 | 16.3 | 109.4 | 1286 | 15.62 | 21.26 | 0.25 |
| alfa | 0.33 | 11.7 | 23.6 | 68.0 | 1256 | 5.72 | 7.03 | 0.11 |
| | 0.10 | 13.4 | 28.2 | 44.4 | 1021 | 3.00 | 3.53 | 0.02 |
| 40k-HEP- | 1.00 | 10.3 | 17.7 | 104.8 | 1245 | 14.32 | 17.85 | 0.26 |
| [O]-N8 | 0.33 | 12.4 | 23.9 | 67.5 | 1211 | 5.91 | 7.12 | 0.08 |
| | 0.10 | 14.2 | 28.7 | 45.6 | 1012 | 3.16 | 3.51 | 0.03 |
| none | 0.00 | 16.4 | 42.8 | 17.7 | — | 0.67 | 0.78 | 0.01 |

### Example 28

### Pharmacokinetics of 40k-HEP-[O]-N8 after i.v. administration to F8-KO mice

A pharmacokinetic study was performed to evaluate the single dose pharmacokinetics and dose-proportionality of 40K-HEP-[0]-N8 in factor 8 knock-out (F8-KO) mice. Forty-eight (48) F8-KO mice (B6.129S4-F8tm1Kaz/J, exon 16 disrupted, bred at Taconic M&B) with a mean weight of app. 22 g were dosed intravenously in a tail vein with a single dose 280, 140, 70 or 35 U/kg (5 ml/kg) of 40k-HEP-[O]-N8. Blood was sampled from the orbital plexus in a sparse sample schedule with n=4 at each time point and three samples from each mouse in the time range of 0.08 and 65 h post administration. Blood was stabilised in 0.13 M sodium citrate (9:1) and diluted 1:4 with a FVIII Coatest SP buffer (50 mM TRIS-HCl, 1% BSA, Ciprofloxacin 10 mg/L, pH 7.3) and centrifuged at room temperature, 4000 g for 5 min. Plasma was kept at −80° C. prior to analysis by means of FVIII chromogenic activity and FVIII antigen based Luminescent Oxygen Channeling Immunoassay (LOCI).

The FVIII chromogenic activity assay was analysed using Coatest® SP FVIII, Chromogenix (#82 4086 63). Calibration was done using N8 SRM (Internal Novo Nordisk FVIII reference material, batch 307.7008.09.2) diluted in FVIII coatest SP buffer to produce calibrators in the range 0-5.0 mU/ml. Plasma samples was diluted 1:80, 1:240, 1:720 and 1:2160 and different dilutons of control plasma N (ORKE 41, Siemens Health care diagnostics product GmbH) were included as quality controls.

The FVIII antigen based LOCI assay was essentially build as the human insulin LOCi described by Poulsen, F & Jensen KB, J Biomol screen 2007; 12(2):240-7. The two antibodies used was in-house produced Novo Nordisk monoclonal anti-rFVIII 4F11 and 4F45.

Results were analysed by means of non-compartmental analysis (NCA) using Phoenix WinNonlin (version 6.3, Pharsight).The FVIII chromogenic activity versus time profile or the FVIII antigen concentration versus time profile after iv administration of 40K-HEP-[O]-N8 seemed to follow a single phase log-linear relationship in the studied time interval, reflecting a minor contribution of initial distribution. The mean estimated half-life of 40K-HEP-[O]-N8 was 14.0 h, and the mean clearance and volume of distribution was estimated to 3.9 ml/h/kg and 78 ml/kg, respectively, based on FVIII chromogenic activity (Table 5). When dose was increased a proportional increase in plasma concentrations were observed. The estimated clearance was approximately the 2-fold reduced, and the half-life of 40K-HEP-[O]-N8 was approximately 2-fold larger than previously published for turoctocog alfa (N8, CI=8.1 ml/h/kg, t½=6.8 h, MRT 9.7 h, Stennicke et al, Blood, 14, 2013, Vol 121:11)

TABLE 5
Estimated pharmacokinetic parameters based on FVIII chromogenic
activities after i.v. administration of 40K-HEP-[O]-N8 in four dose
levels to F8-KO mice

| I.v. dose | T_{1/2} | Cₘₐₓ | CL | MRT | Vₛₛ |
|---|---|---|---|---|---|
| (U/kg) | (h) | (U/mL) | (ml/h/kg) | (h) | (ml/kg) |
| 280 | 15.3 | 3.6 | 3.5 | 22 | 77 |
| 140 | 14.2 | 1.94 | 3.7 | 20 | 75 |
| 70 | 11.0 | 0.97 | 4.5 | 16 | 70 |
| 35 | 15.5 | 0.42 | 4.0 | 22 | 90 |
| Mean | 14.0 | — | 3.9 | 20 | 78 |

### Example 29

### Pharmacokinetics of 40k-HEP-[O]-N8 after i.v. Administration to Rats

A pharmacokinetic study in four (4) male Wistar rats (Taconic, app. 250 g) was performed. The rats were dosed intravenously in a tail vein with a single dose 250 U/kg of 40k-HEP-[O]-N8 and blood was sampled from another tail vein at predose 0.08, 1, 4, 7, 24, 30, 48 h post administration (full profiles, n=4). Blood was stabilised in

0.13 M sodium citrate (9:1) and diluted 1:4 with a FVIII Coatest SP buffer (50 mM TRIS-HCl, 1% BSA, Ciprofloxacin 10 mg/L, pH 7.3) and centrifuged at room temperature, 4000 g for 5 min. Plasma was kept at −80° C. prior to analysis by means of FVIII chromogenic activity and FVIII antigen based Luminescent Oxygen Channeling Immunoassay (LOCI) (assay descriptions see Example 28). A baseline value (predose samples) was obtained in the FVIII chromogenic activity assay with a mean±SD of 0.42±0.17 U/ml. All FVIII chromogenic activity data was baseline subtracted prior to were analysis by means of non-compartmental analysis (NCA) using Phoenix WinNonlin (version 6.3, Pharsight). The mean clearance of 40k-HEP-[O]-N8 and mean volume of distribution was estimated to 3.1 ml/h/kg and 51 ml/kg, respectively, based on FVIII chromogenic activity after i.v. administration to Wistar rats. The mean half-life of 40k-HEP-[O]-N8 was estimated to 12 h based on FVIII chromogenic activity. This corresponds to an approximately 2-fold prolongation in half-life, as the clearance and half-life of recombinant FVIII after i.v. administration to rats was previously reported to 9.4 ml/h/kg and 5.8 h, respectively (Stennicke et al, Blood, 14, 2013, Vol 121:11).

### Example 30

### Pharmacokinetics of 40k-HEP-[O]-N8 after i.v. Administration to Cynomolgus Monkeys

A pharmacokinetic study in three (3) male Cynomolgus monkeys (Macaca fascicularis, Bioculture (Mauritius) Ltd, Mauritius, app. 3 kg) was performed. Monkeys were i.v. administered 40K-HEP-[0]-N8 250 U/kg via a saphenous veins and 0.9 ml blood was withdrawn from femoral vein/artery into 0.1 ml 0.13 M trisodium citrate anticoagulant at predose, 0.25, 2, 6, 12, 24 and 48 post administration. The sample was mixed gently by hand then continuously for at least 1 minute on an automatic mixer. The sample was centrifuged within 10 minutes for 5 minutes at 2000 g at room temperature, and plasma stored at −80° C. prior to analysis of FVIII antigen based FVIII Luminescent Oxygen Channeling Immunoassay (LOCI) and FVIII chromogenic activity (assay descriptions see Example 28). All data was baseline subtracted prior to PK analysis by means of non-compartmental analysis (NCA) using Phoenix WinNonlin (version 6.3, Pharsight).

The mean clearance of 40k-HEP-[O]-N8 and mean volume of distribution was estimated to 1.13 ml/h/kg and 32 ml/kg, respectively, based on FVIII chromogenic activity after i.v. administration to cynomolgus monkeys. The mean half-life of 40k-HEP-[O]-N8 was estimated to 20 h based on FVIII chromogenic activity. This corresponds to an approximately 2-fold prolongation in half-life of 40k-HEP-[O]-N8, as the clearance and half-life of turoctocog alfa after i.v. administration to cynomolgus monkeys was previously reported to 8.3 ml/h/kg and 5.4 h, respectively (Stennicke et al, Blood, 14, 2013, Vol 121:11).

TABLE 6
Chromogenic FVIII activity data: Pharmacokinetic parameters estimated
by means of non-compartmental analysis (NCA) of the predose-subtracted
chromogenic activity values after i.v. administration of 250 U/kg
40K-HEP-[O]-N8 to cynomolgus monkeys(mean ± SD, n = 3)

| Parameter | 40k-HEP-[O]-N8 |
|---|---|
| Dose | 250 U/kg |
| Cₘₐₓ (U/L) | 8349 ± 553 |
| T½ (h) | 20 ± 1.9 |
| Cl (mL/kg *h) | 1.13 ± 0.08 |
| V (mL/kg) | 32 ± 3.1 |
| MRT (h) | 28 ± 2.4 |

### Example 31

### The Dose Response of 40k-HEP-[O]-N8 in the Tail Vein Transection (TVT) Model in F8-KO Mice

A dose response effect study of 40k-HEP-[O]-N8 were performed in the TVT bleeding model in isoflurane anaesthetised F8-KO mice (B6.129S4-F8tm1Kaz/J, exon 16 disrupted, bred at Taconic M&B). 4 groups of 12 mice each were dosed 40k-HEP-[0]-N8 in doses of 0 (vehicle), 0.25, 1, and 4 U/kg (5 ml/kg) in the right lateral tail vein 5 minutes prior to TVT. After injury, the tail was placed in saline at 37° C. The blood was collected for 60 minutes, and the blood loss was quantified by analysis of haemoglobin. ED₅₀ values of the blood loss were estimated by fitting an inverse dose response equation to the data.

The blood losses were 5482±663, 6117±573, 2754±611, and 1782±423 nmol haemoglobin for 40k-HEP-[O]-N8 in the groups treated with 0 (vehicle), 0.25, 1, and 4 U/kg. The blood loss at the highest dose level (4 U/kg) differed from the vehicle group. The ED₅₀ and 95% CI was estimated to 1.4 U/kg [0.3-7 U/kg] for 40k-HEP-[O]-N8.

### Example 32

### The Duration of Effect of 40k-HEP-[O]-N8 in the TVT Model in F8-KO Mice

A study of the duration of effect was performed after i.v. administration of 40k-HEP-[O]-N8 in a tail vein transection (TVT) bleeding model in isoflurane anaesthetised F8-KO mice (B6.129S4-F8tm1Kaz/J, exon 16 disrupted, bred at Taconic M&B).

40k-HEP-[O]-N8 were injected in the right lateral tail vein in a dose of 10 U/kg at 24, 48, or 72 h (5 ml/kg) prior to TVT (n=12). The vehicle group comprised 12 mice total; 4 mice at each time point. Blood was collected for a total of 60 minutes while the tail was immersed in pre-heated saline at 37° C. Blood loss was determined by haemoglobin concentration in the collected blood.

Blood losses at 24, 48, and 72 hours were 526±145, 1919±558, and 4686±648 nmol haemoglobin for 40k-HEP-[O]-N8 (mean±SEM). The mean blood loss in the vehicle group was 7269±258 nmol haemoglobin.

40k-HEP-[O]-N8 was haemostatically active at all studied time points and the effect decreased following the expected elimination from the circulation.

### Example 33

### Pharmacokinetics and Ex Vivo Pharmacodynamics of 40k-HEP-[O]-N8 and N8-GP in Haemophilia A Dogs

A study of the pharmacokinetics and ex vivo pharmacodynamics was evaluated in 4 haemophilia A dogs (colony of the Blood Research Laboratory (FOBRL, University of North Carolina, Chapel Hill). The dogs were infused iv over 10 min with 125 U/kg of 40k-HEP-[O]-N8. Whole blood samples were drawn pre-infusion and at different time points over six days. One part of the whole blood samples was centrifuged and plasma aliquoted for later measurements of FVIII concentrations using FVIII chromogenic activity as described in Example 28. Pharmacokinetic parameters were estimated using a non-compartmental analysis (NCA) using Phoenix WinNonlin (version 6.3, Pharsight). Another part of the unstablised whole blood samples was analysed immediately after sampling by measuring whole blood clotting time (WBCT) and thrombelastography (TEG, data not included).

WBCT was performed by a two-tube procedure at 28° C. as previously described (Nichols T C et al. ILAR J 2009; 50(2): 144-67). Briefly, one ml of whole blood was collected with a 1 mL syringe and was distributed equally between two siliconised tubes (Vacutainer™; Becton-Dickinson, Franklin Lakes, N.J., USA). The first tube was tilted every 30 s after an initial incubation of 1 min. After formation of the clot, the second tube was tilted and was observed every 30 s. The endpoint was the clotting time of the second tube. The ex vivo effect profiles were analysed by a random coefficient linear regression model.

TABLE 7
Estimated pharmacokinetic parameters on the FVIII activity
concentration vs time data after infusion of administration of
125 U/kg to haemophilia A dogs. Mean ± SEM (n = 4).

| Parameter | 40k-HEP-[O]-N8 |
|---|---|
| Dose (U/kg) | 125 |
| Cₘₐₓ (U/mL) | 2.49 ± 0.13 |
| T_{1/2} (h) | 15.2 ± 1.7 |
| Cl (mL/kg *h) | 2.51 ± 0.39 |
| V (mL/kg) | 52.3 ± 3.1 |
| MRT (h) | 21.4 ± 2.5 |

The pre-infusion WBCT were measured to 32.0±2.1 min for 40k-HEP-[O]-N8. Immediately after the infusion (t=5 min), WBCT were normalized, being 10.8±1.0 min for 40k-HEP-[O]-N8. Thereafter, WBCT gradually increased over time towards the haemophilic phenotype, with an estimated slope of to 0.079 min*h⁻¹ for 40k-HEP-[O]-N8.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A conjugate comprising a Factor VIII polypeptide, a linking moiety, and a heparosan polymer, wherein the linking moiety between the Factor VIII polypeptide and the heparosan polymer comprises X as follows:
[heparosan polymer]-[X]-[Factor VIII]
wherein X comprises a sialic acid derivative connected to a moiety according to Formula 1 below:

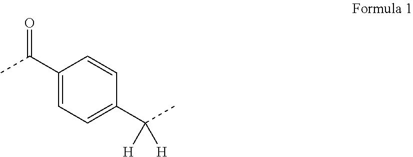

2. The conjugate according to claim 1, wherein the sialic acid derivative is a sialic acid derivative according to Formula 2 below: 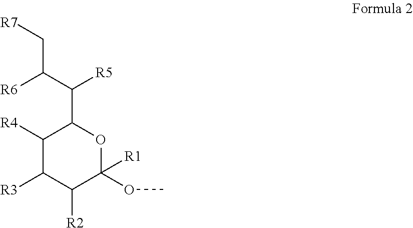
wherein R1 is selected from —COOH, —CONH₂, —COOMe, —COOEt, —COOPr and R2, R3, R4, R5, R6 and R7 independently can be selected from —H, —NH₂, —SH, —N3, —OH, —F.

3. The conjugate according to claim 1, wherein the sialic acid derivative is a glycyl sialic acid according to Formula 3 below: 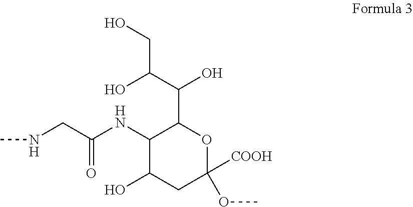
and wherein the moiety of Formula 1 is connected to the terminal —NH handle of Formula 3.

4. The conjugate according to claim 1, wherein
[heparosan polymer]-[X]-
comprises the structural fragment shown in Formula 4 below: 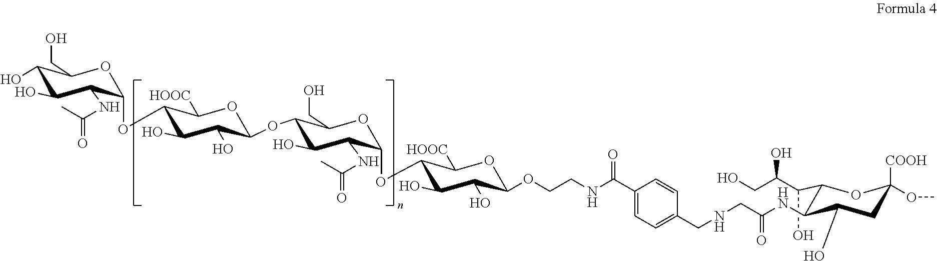
wherein n is an integer from 5 to 450.

5. A conjugate according to claim 1, wherein FVIII is a B domain truncated FVIII molecule, wherein the sequence of the truncated B domain is selected from the group consisting of SEQ ID NO:2, SEQ ID NO:3, and SEQ ID NO:4.

6. A conjugate according to claim 1, wherein the molecular weight of the heparosan polymer is 5-150 kDa.

7. A conjugate according to claim 1, wherein the molecular weight of the heparosan polymer is 35-45 kDa.

8. A conjugate according to claim 1, wherein the molecular weight of the heparosan polymer is 40 kDa+/−10%.

9. A conjugate according to claim 1, wherein the heparosan polymer is conjugated to FVIII via an O-linked glycan in the B domain, wherein FVIII activation results in removal of said heparosan polymer.

10. A conjugate according to claim 1, wherein said heparosan polymer is linked to FVIII via an O-linked glycan attached to a serine amino acid residue corresponding to the Ser750 residue in SEQ ID NO: 1, and wherein the link between FVIII and heparosan comprises the following structure:

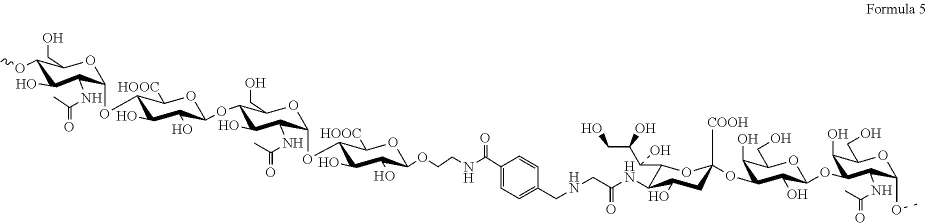

11. A pharmaceutical composition comprising a conjugate according to claim 1.

12. Use of a conjugate according to claim 1 for reducing inter-assay variability in aPTT-based assays.

13. A conjugate according to claim 1 for use as a medicament.

14. A conjugate according to claim 1 for use in treatment of haemophilia.

15. A method of conjugating a heparosan polymer to a FVIII polypeptide comprising the steps of:
(i) reacting a heparosan polymer comprising a reactive amine [HEP-NH] with an activated 4-formylbenzoic acid to yield the compound of Formula 6 below, 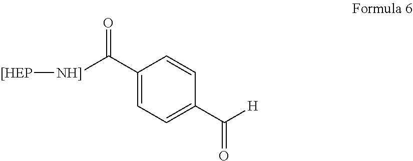
wherein said reactive amine may be directly attached to the heparosan polymer or attached via a linking moiety connecting the reactive amine with said heparosan polymer,
(ii) reacting the compound of Formula 6 with a CMP-activated sialic acid derivative under reducing conditions,
(iii) conjugating the compound obtained in step (ii) to a glycan on the Factor VIII polypeptide.

16. Conjugates obtainable using the method according to claim 15.


SEQUENCE LISTING (4 sequences)

SEQ ID NO: 1 - protein, 2332 aa, homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 21 aa, artificial
  21 aa B domain linker sequence
SFSQNSRHPSQNPPVLKRHQR

SEQ ID NO: 3 - protein, 20 aa, artificial
  20 aa B domain linker sequence
SFSQNSRHPSQNPPVLKRHQ

SEQ ID NO: 4 - protein, 20 aa, artificial
  20 aa b domain lilnker sequence
FSQNSRHPSQNPPVLKRHQR